(19) United States
(12) Patent Application Publication
(10) Pub. No.: US 2025/0263668 A1
(43) Pub. Date: Aug. 21, 2025

(54) METHODS FOR OBTAINING ANTIBODY PRODUCING CELLS

(71) Applicant: Regeneron Pharmaceuticals, Inc., Tarrytown, NY (US)

(72) Inventors: Wen-Yi LEE, New Hyde Park, NY (US); Kevin BROWN, Mount Vernon, NY (US); Gang CHEN, Yorktown Heights, NY (US)

(21) Appl. No.: 19/053,593

(22) Filed: Feb. 14, 2025

Related U.S. Application Data
(60) Provisional application No. 63/554,374, filed on Feb. 16, 2024.

(51) Int. Cl. C12N 5/071; G01N 33/58

(52) U.S. Cl. CPC C12N 5/0682; G01N 33/58; C12N 2510/02; C12N 2510/04

(57) ABSTRACT

Disclosed herein are methods for detecting cells expressing antibody molecules on the cell surface that bind to a targeted antigen without competing with one or more binding partners of the target antigen. Also disclosed herein are methods for detecting and differentiating cells expressing antibody molecules on the cell surface to a target antigen that are non-competing with one or more binding partners, from cells expressing antibody molecules to a target antigen that block binding by a binding partner. The methods disclosed herein allow for enrichment and isolation of cells expressing antibody molecules on the cell surface to a target antigen that are non-competing with one or more binding partners, and cells expressing antibody molecules on the cell surface to a target antigen that block binding by a binding partner.

### CROSS REFERENCE TO RELATED APPLICATION

This application claims the benefits of priority from U.S. Provisional Application 63/554,374, filed Feb. 16, 2024, the entire contents of which are incorporated herein by reference.

### BACKGROUND

Monoclonal antibodies (mAbs) are valuable biomolecules and have well established applications in prognosis, diagnosis, and therapeutics in various diseases including cancer and infectious diseases. B cell sorting has been developed for high-throughput screening and isolation of single B cells that express monoclonal antibodies. A need exists for processes of sorting B cells and other antibody-producing cells that permit enrichment and isolation of cells that express antibody molecules on the cell surface that have unique and desirable features.

### SUMMARY

Disclosed herein are methods for detecting cells expressing antibody molecules that bind to a targeted antigen without competing with one of more binding partners of the target antigen. The target antigen can be a monomeric antigen or a multimeric antigen. Binding partners of a target antigen, also referred to as binders herein, can be an antibody or an antigen-binding fragment thereof, or a receptor or a ligand for the target antigen. Also disclosed herein are methods for detecting and differentiating cells expressing antibody molecules to a target antigen that are non-competing with one or more binding partners, from cells expressing antibody molecules to a target antigen that block binding by a binding partner. The methods disclosed herein allow for enrichment and isolation of cells expressing antibody molecules to a target antigen that are non-competing with one or more binding partners, and cells expressing antibody molecules to a target antigen that block binding by a binding partner.

In a first aspect, the present disclosure is directed to a method for obtaining antibody-producing cells that express antibody molecules to a monomeric target antigen that are non-competitive with a first binder to the target antigen, the method comprising:
(a) contacting a population of antibody-producing cells with:
   (1) the target antigen; and
   (2) the first binder to the target antigen, wherein the first binder is conjugated with a detectable label;
   to permit binding of cells that express antibody molecules that are non-competitive with the first binder to bind to the target antigen; and
(b) collecting cells that are bound to the target antigen which is bound by the first binder conjugated to the detectable label, thereby obtaining a population of antibody-producing cells expressing antibody molecules that are non-competitive with the first binder.

In some embodiments, the target antigen in step (a) is prebound with the first binder prior to being contacted by the population of antibody-producing cells.

In some embodiments, the target antigen in step (a) is not prebound with the first binder, but is prebound with at least one other binder that does not compete with the first binder in binding to the target antigen, and the contacting permits cells that express antibody molecules that are non-competitive with the first binder and non-competitive with the at least one other binder to bind to the target antigen. In some embodiments, the at least one other binder is conjugated to a detectable label, wherein the detectable label can be the same or different from the detectable label conjugated to the first binding. In some embodiments, the at least one other binder comprises two or more binders that do not compete with the first binder; and in some such embodiments, the two or more binders are each conjugated to a detectable label. The detectable labels conjugated to the two or more binders can be the same or different from each other, and can be the same or different from the detectable label conjugated to the first binder. In some embodiments, the target antigen is not prebound with any binder prior to contacting the population of antibody-producing cells. In some embodiments, the cells are contacted with the target antigen, and subsequently with the first binder conjugated with a detectable label, optionally with at least one other binder which may or may not be conjugated with a detectable label.

In some embodiments, the method further comprises a wash step between contacting with the target antigen and contacting with the first binder. In some embodiments, the wash step is repeated at least once. In some embodiments, the method further comprises a wash step after step (a) but before collecting cells in step (b). In some embodiments, the wash step after step (a) and before the collecting step (b) is repeated at least once.

In some embodiments, the first binder is an antibody or antigen binding fragment thereof. In some embodiments, the first binder is a receptor or ligand-binding domain thereof (and the target antigen is a ligand for the receptor). In some embodiments, the first binder is a ligand (and the target antigen is a receptor for the ligand). In some embodiments, at least one other binder is an antibody or antigen binding fragment thereof. In some embodiments, the at least one other binder comprises two or more binders each being an antibody or antigen binding fragment thereof.

In some embodiments, the detectable label conjugated to the first binder is a fluorescent label. In some embodiments, the detectable label conjugated to the first binder is Alexa647. In some embodiments, the detectable label conjugated to the first binder is a biotin molecule. In some embodiments, the detectable label conjugated to the at least one other binder or other binders is a fluorescent label. In some embodiments, the detectable label conjugated to the at least one other binder or other binders is Alexa647. In some embodiments, the detectable label conjugated to the at least one other binder or other binders is a biotin molecule.

In some embodiments, step (a) further comprises, after the population of antibody producing cells are contacted with the target antigen which is bound with the first binder and conjugated with a biotin to permit binding of cells that express antibody molecules that are non-competitive with the first binder to bind to the target antigen, washing the cells and contacting the cells with streptavidin conjugated with a fluorescent label.

In some embodiments, the target antigen is a protein. In some embodiments, the protein is an external glycoprotein of a virus. In some embodiments, the protein is a SARS-COV-2 RBD (receptor binding domain) protein, an Ebola glycoprotein subunit (monomer), MERS-COV RBD monomer protein, pfCSP (i.e. Malaria sporozoite surface protein); or Proprotein convertase subtilisin/kexin type 9 (PCSK9) (which binds to the EGF-like repeat (EGF-A) domain of low-density lipoprotein receptors (LDLR)).

In some embodiments, the antibody-producing cells are contacted with the target antigen at a concentration from 0.02 nM to 25 nM. In some embodiments, the antibody-producing cells are contacted with the first binder conjugated with a detectable label at a concentration from 0.01 nM to 100 nM.

In some embodiments, the antibody producing cells are mammalian cells or yeast (such as S. cerevisiae or Pichia) that produce antibody molecules on the cell surface. In some embodiments, the antibody producing mammalian cells are primary antibody producing cells. In some embodiments, the antibody producing cells are immortalized cells which produce antibody molecules on the cell surface. In some embodiments, the primary antibody-producing cells are obtained from the spleen, lymph node, peripheral blood and/or bone marrow of a mammal. In some embodiments, the immortalized cells are chosen from Chinese hamster ovary (CHO) cells, Human Embryonic Kidney (HEK) 293 cells, and hybridoma cells which produce antibody molecules on the cell surface.

A second aspect of the current disclosure is directed to a method for obtaining antibody-producing cells that express antibody molecules to a multimeric target antigen that are non-competitive with a first binder to the multimeric target antigen, the method comprising:
(a) contacting a population of antibody-producing cells with the target antigen, wherein the target antigen is conjugated with a detectable label and is prebound by the first binder to permit binding of cells that express antibody molecules that are non-competitive with the first binder to bind to the target antigen; and
(b) collecting cells that are bound to the target antigen that is conjugated with the detectable label.

In some embodiments, the target antigen is prebound by a first binder and at least one other binder, and the cells collected are cells that express antibody that does not compete with either the first binder or the at least one other binder. In such embodiments, the method comprises:
(a) contacting a population of antibody-producing cells with a target antigen, wherein the target antigen is conjugated with a detectable label and is prebound by a first binder and at least one other binder, to permit binding of cells that express antibody molecules that are non-competitive with either the first binder or the at least one other binder in binding to the target antigen; and
(b) collecting cells that are bound to the target antigen that is conjugated with the detectable label.

In some embodiments, the method further comprises a wash step after step (a) but before collecting cells in step (b). In some embodiments, the wash step after step (a) and before the collecting step (b) is repeated at least once. In some embodiments, the first binder is an antibody or antigen binding fragment thereof. In some embodiments, the first binder is a receptor or a ligand.

In some embodiments, the antibody-producing cells are contacted with the target antigen at a concentration from 0.02 nM to 25 nM.

In some embodiments, the concentration of the first binder is in excess (e.g., 5-10 fold to 100 fold) relative to the concentration of the target antigen. In embodiments where the antigen is also prebound to at least one other binder, the at least one other binder is also in excess (e.g., 5-10 fold to 100 fold) relative to the concentration of the target antigen.

In some embodiments, the detectable label conjugated to the target antigen is a biotin label. In some embodiments, the detectable label conjugated to the target antigen is a fluorescent label. In some embodiments, the detectable label conjugated to the target antigen is Alexa647.

In some embodiments, step (a) further comprises, after the population of antibody producing cells are contacted with the target antigen prebound with the first binder (and optionally with at least one other binder) and conjugated with a biotin to permit binding of cells that express antibody molecules that are non-competitive with the first binder (or with at least one other binder) to bind to the multimeric target antigen, washing the cells and contacting the cells with streptavidin conjugated with a fluorescent label.

In some embodiments, the multimeric target antigen is a protein. In some embodiments, the protein is an external glycoprotein of a virus. In some embodiments, the protein is an Ebola glycoprotein, an Influenza HA trimer protein, a SARS-COV-1 Spike trimer protein, or a MERS-COV Spike trimer protein.

In some embodiments, the antibody producing cells are mammalian cells or yeast (such as S. cerevisiae or Pichia) that produce antibody molecules on the cell surface. In some embodiments, the antibody producing mammalian cells are primary antibody producing cells. In some embodiments, the antibody producing cells are immortalized cells which produce antibody molecules on the cell surface. In some embodiments, the primary antibody-producing cells are obtained from the spleen, lymph node, peripheral blood and/or bone marrow of a mammal. In some embodiments, the immortalized cells are chosen from Chinese hamster ovary (CHO) cells, Human Embryonic Kidney (HEK) 293 cells, and hybridoma cells which produce antibody molecules on the cell surface.

A third aspect of the current disclosure is directed to a method for obtaining antibody-producing cells that express antibody molecules to a monomeric target antigen that are competitive and/or non-competitive with a first binder to the target antigen, the method comprising:
(a) contacting a population of antibody-producing cells with:
   (1) the target antigen conjugated to a first detectable label; and
   (2) the first binder to the target antigen, wherein the first binder is conjugated with a second detectable label that is different from the first detectable label; to permit binding of cells that express antibody molecules that are non-competitive with the first binder to bind to the target antigen; and
(b) collecting cells that are bound to:
   (1) the target antigen conjugated to the first detectable label but not the first binder conjugated with the second detectable label, thereby obtaining a population of antibody-producing cells expressing antibody molecules that compete with the first binder in binding to the target antigen; and/or
   (2) the target antigen conjugated to the first detectable label and the first binder conjugated to the second detectable label thereby obtaining a population of antibody producing cells expressing antibody molecules that are non-competitive with the first binder in binding to the target antigen.

In some embodiments, the target antigen conjugated to the first detectable label in step (a)(1) is prebound with the first binder conjugated to the second detectable label of (a)(2), prior to contacting the population of antibody-producing cells. In some embodiments, in step (a), the population of antibody-producing cells is first contacted with the target antigen conjugated to the first detectable label, and subsequently contacted by the first binder conjugated to the second detectable label. In some embodiments, the target antigen conjugated to a first detectable label is prebound with at least one other binder before the step of contacting the population of antibody-producing cells with the target antigen conjugated to the first detectable label, and wherein the at least one other binder does not compete with the first binder. In some embodiments, the at least one other binder comprises two or more binders that do not compete with the first binder.

In some embodiments, the first binder is an antibody or antigen binding fragment thereof. In some embodiments, the first binder is a receptor or ligand binding domain thereof, or is a ligand. In some embodiments, the at least one other binder is an antibody or antigen-binding fragment thereof. In some embodiments, the first binder is in a multiple valent form. In some embodiments, the multivalent form is provided by a multimer (e.g., streptavidin or foldon) pre-clustered with the first binder. In some embodiments, the at least one other binder is conjugated to a third detectable label. In some embodiments, the at least one other binder comprises two or more binders each being conjugated to a detectable label, where the detectable labels conjugated to the two or more binders can be the same or different from each other and can be the same or different from the second detectable label conjugated to the first binder.

In some embodiments, the antibody-producing cells are contacted with the target antigen at a concentration from 0.02 nM to 25 nM.

In some embodiments, the antibody-producing cells are contacted with the first binder at a concentration from 0.01 nM to 100 nM.

In some embodiments, the first detectable label and the second detectable label are two different fluorescent labels. In some embodiments, the third detectable label if present, and the first detectable label, are two different fluorescent labels. In some embodiments, the third detectable label if present, and the second detectable label, can be the same or different fluorescent labels. In some embodiments, the first, second and third detectable labels are all fluorescent labels, wherein the second and third detectable labels are the same and differ from the first detectable label. In some embodiments, the first, second and third detectable labels are all fluorescent labels, wherein all differ from each other.

In some embodiments, the antigen prebound by the first binder is a protein.

In some embodiments, the antibody producing cells are mammalian cells or yeast (such as S. cerevisiae or Pichia) that produce antibody molecules on the cell surface. In some embodiments, the antibody producing mammalian cells are primary antibody producing cells. In some embodiments, the antibody producing cells are immortalized cells which produce antibody molecules on the cell surface. In some embodiments, the primary antibody-producing cells are obtained from the spleen, lymph node, peripheral blood and/or bone marrow of a mammal. In some embodiments, the immortalized cells are chosen from Chinese hamster ovary (CHO) cells, Human Embryonic Kidney (HEK) 293 cells, and hybridoma cells which produce antibody molecules on the cell surface.

A fourth aspect of this disclosure is directed to a method comprising isolating antibody-encoding nucleic acids from the cells collected in step (b) according to any of the methods described above.

In some embodiments, the method further comprises transfecting a host cell with a nucleic acid encoding an antibody heavy chain or variable domain thereof, and a nucleic acid encoding an antibody light chain or variable domain thereof; and growing the transfected host cell under conditions to support expression of antibody by the host cell. In some embodiments, the host cell is Chinese hamster ovary (CHO) cell.

A fifth aspect of the disclosure is directed to a mammalian host cell made by isolating antibody-encoding nucleic acids from the collected cells of the methods disclosed herein, and transfecting a host cell with a nucleic acid encoding an antibody heavy chain or variable domain thereof, and a nucleic acid encoding an antibody light chain or variable domain thereof; and growing the transfected host cell under conditions to support expression of antibody by the host cell. In some embodiments, the host cell is a CHO cell.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1A illustrates an exemplary embodiment of a direct antigen sort method for obtaining cells expressing antibody specific to a monomeric antigen. In this embodiment, antibody-producing cells (such as B cells, yeast, hybridoma cells, CHO cells or other immortalized cells) are contacted with a biotin labeled monomeric antigen. After removing unbound biotin labeled monomer, cells that are bound to the biotin labeled monomer can further be contacted with a fluorophore (e.g., Phycoerythrin (PE)) labeled streptavidin tetramer that forms complex with the biotin conjugated to the monomer bound to the cells. Cells labeled with the fluorophore can be collected and are cells that express antibody specific to the monomeric antigen.

FIG. 1B illustrates an exemplary embodiment of a non-competing sort method for obtaining cells expressing antibody that is specific to a monomeric antigen and does not compete with one or more antibody molecules (such as two antibodies as shown). In this embodiment, antibody producing cells (such as B cells, yeast, hybridoma cells, CHO cells or other immortalized cells) are contacted with an unlabeled monomer that is pre-bound to unlabeled Fab of Antibody 1. After removing the unbound monomer, the antibody-producing cells bound to the unlabeled monomer which is pre-bound with unlabeled Fab of Antibody 1 are further contacted with fluorophore (e.g., Alexa fluor 647) labeled Antibody 2 in the presence of excessive unlabeled Fab of Antibody 1. Antibody 1 and Antibody 2 do not compete with each other in binding to the monomer. Cells are then washed further in phosphate buffered saline (PBS) to remove unbound complex. Cells labeled with the fluorophore can be collected and these are cells that express antibody that is specific to the monomeric antigen and does not compete with Antibody 1 or Antibody 2.

FIG. 2A-2B are representative staining profiles that demonstrate the detection and separation of antibody-expressing B cells using the direct antigen sort method depicted in FIG. 1A. Cells were analyzed by flow cytometry as described below: A population of B cells (i.e., positive surface IgG staining cells) that expressed antibody specific to spike protein RBD monomer was detected and collected using FACS by staining the splenocytes with fluorescent labels to B cell markers (i.e., anti-IgG) and contacting the population of cells with the biotin labeled spike protein RBD monomer. FIG. 2A showed that only four B cells in one million splenocytes obtained from the mouse bound non-specifically to a biotin labeled irrelevant protein that fell into the (rectangle). FIG. 2B showed that one hundred and eighty-five B cells out of one million splenocytes obtained from the mouse expressing an antibody specific to the spike protein RBD monomers were detected (rectangle).

FIG. 2C-2D are representative staining profiles that demonstrate the detection and separation of antibody-expressing B cells using the non-compete sort method depicted in FIG. 1B. Cells were analyzed by flow cytometry as described below: A sub-population of B cells (positive surface IgG staining cells) that expressed antibody specific to spike protein RBD monomer was detected and collected using FACS by staining the solenocytes with fluorescent labels to B cell markers (i.e., anti-IgG) and contacting the cells with the unlabeled spike protein RBD monomer that was pre-bound with Fab of antibody R001, known to bind SARS RBD monomer protein. Cells were further contacted by Alexa fluor 647-labeled antibody (R002, known to bind SARS RBD monomer protein and is not competitive with R001) and detected by Alexa647 signal emitted by the complex after washing off the unbound. FIG. 2C showed that only two B cells in one million splenocytes were obtained from the mouse bound non-specifically to an unlabeled irrelevant protein that was further contacted with Alexa flour 647-labeled antibody R002 (rectangle). FIG. 2D showed that sixty-seven B cells out of one million splenocytes obtained from the mouse expressing an antibody specific to the unlabeled spike protein RBD monomer that was pre-bound with Fab of R001 and further contacted with Alexa 647-labeled R002 were detected (rectangle).

FIG. 3A demonstrates Luminex bindings results from the B cells collected from three SARS-CoV-2 RBD immunized mice using the direct antigen sort method depicted in FIG. 1A as well as the non-competing sort method depicted in FIG. 1B. For the clones obtained from the direct antigen sort method, three hundred and five clones were tested with 2019-n-CoV-2 RBD.mmh protein, and with irrelevant FelD1.mmh protein. For the clones obtained from non-competing sort method, two hundred and sixty-three clones were tested with the same two proteins. Here, both methods showed comparable antigen-specific binding percentages to 2019-n-CoV-2 RBD.mmh: 91.8% of the tested clones from the direct antigen sort method have binding mean fluorescence intensity (MFI) greater than 1,000 and are deemed as antigen-specific; 83.2% of the tested clones from non-competing sort method have binding MFI greater than 1,000. None of the clones from both methods showed specific binding to the irrelevant protein (>MFI 1,000).

FIG. 3B displays the calculated percentage inhibition of antibody R001 on the individual tested anti-SARS-CoV-2 RBD monoclonal antibodies isolated using either the direct antigen sort method depicted in FIG. 1A or the non-compete sort method depicted in FIG. 1B. Out of the two hundred and fifty-five antibodies isolated using the direct antigen sort method, fifty-two clones or 20% showed greater than 50% antigen binding inhibition when RBD was pre-bound with R001; while out of the one hundred and seventy-three antibodies isolated using the non-competing sort method, none (0%) of them showed greater than 50% inhibition in binding to SARS-CoV-2 RBD that prebound with R001.

FIG. 3C demonstrates a similar pattern of the calculated percentage inhibition when RBD is pre-bound with antibody R002. 17% of the antibodies (43 out of 255 clones) isolated using direct antigen sort method showed greater than 50% inhibition in binding to R002-prebound SARS-CoV-2 RBD; while only as low as 4% of the tested anti-SARS-CoV-2 RBD monoclonal antibodies (7 out of 173 clones) isolated through non-compete sort method showed greater than 50% inhibition in binding to R002-prebound SARS-CoV-2 RBD.

FIG. 4A and FIG. 4B depict the strategies designed for proof-of-concept (POC) experiments for obtaining cells expressing antibodies specific to a multimeric antigen such as the Ebola Zaire glycoprotein (GP) using the direct sort method (FIG. 4A) and the non-compete sort method (FIG. 4B). In both FIG. 4A and FIG. 4B, beads are separately coated with four Ebola Zaire GP antibodies with various levels of competition to a known anti-Ebola GP antibody, R003, where R003 and R004 compete binding to Ebola GP protein with R003, while R005 and R006 do not compete binding to Ebola GP protein with R003. In the direct sort method as shown in FIG. 4A, beads are first separately coated with the four antibodies. Antibody-coated beads are contacted with biotin labeled Ebola GP trimer protein. After washing off the unbound, beads are further contacted with a fluorophore (e.g., PE) labeled streptavidin tetramer that forms complex with the biotin on the Ebola GP trimer protein bound to the beads. Beads are then scanned and detected by a fluorescence-based method like Flow Cytometry. In the non-competing sort method shown in FIG. 4B, antibody-coated beads are contacted with biotin labeled Ebola GP trimer protein that is pre-incubated with excessive Fab of R003. After washing off the unbound, beads are further contacted with a fluorophore (e.g., PE) labeled streptavidin tetramer that forms complex with the biotin on the Ebola GP trimer protein in the presence of excessive Fab of R003. Beads are further scanned and detected by a fluorescence-based method like Flow Cytometry.

FIGS. 5A and 5B are modeled overlays of PE staining dot plots showing the anticipated results from experiments conducted based on the strategies depicted in FIGS. 4A and 4B. In FIG. 5A, R003-competing antibody coated beads are anticipated to exhibit weakened PE staining level (red dots) and moved outside the “Ag+” box when using the non-competing sort method, due to binding epitopes masked by R003 Fab. In FIG. 5B, R003 non-competing antibody coated beads are anticipated to exhibit unchanged high PE staining level regardless of the sort methods used. This is due to that the antibody coated on beads is not competing with R003 binding epitope on the Ebola GP trimer, and the binding should not be affected by prebinding R003 Fabs onto the Ebola GP trimer (red dots).

FIG. 6A and FIG. 6B are representative overlays of PE staining dot plots with R003 coated beads and with R004 coated beads detected by flow cytometry, respectively. R003 and R004 are competing antibodies for R003. Three different samples are shown here: light blue dots are beads contacted with a biotin labeled irrelevant protein (control sample) showing PE negative staining (with MFI at 123 and at 69.5 with R003 and R004 coated beads, respectively); bright blue dots are beads contacted with biotin labeled Ebola GP trimer and further stained with PE labeled streptavidin after removing the unbound (Direct Antigen Sort Method), and have exhibited strong PE signals in the positive gates (rectangular boxes) at MFI 2309 and 3205 with R003 and R004 coated beads, respectively; and red dots are beads exposed with REGN003 Fab-prebound biotin labeled Ebola GP trimer and further stained with PE labeled streptavidin after the removal of the unbound (Non-Competing Sort Method).

FIG. 6C and FIG. 6D are representative overlays of PE staining dot plots with antibody R005 coated beads and with antibody R006 coated beads detected by flow cytometry, respectively. R005 and R006 are non-competing antibodies for R003. Three different samples are shown here: light blue dots are beads incubated with a biotin labeled irrelevant protein (control sample) showing PE negative staining (with MFI at 83.4 and at 56.7 with R005 and R006 coated beads, respectively); bright blue dots are beads contacted with biotin labeled Ebola GP trimer and further stained with PE labeled streptavidin after removing the unbound (direct antigen sort method), and have shown strong PE signals in the positive gates (rectangular boxes) at MFI 3258 and 1433 with R005 and R006 coated beads, respectively; and red dots are beads contacted with R003 Fab-prebound biotin labeled Ebola GP trimer and further stained with PE labeled streptavidin after the removal of the unbound (Non-Competing Sort Method).

FIGS. 7A and 7B illustrates the detection and separation of non-blocking and blocking antibodies for PCSK9 using non-competing method in dual-color format. Proprotein convertase subtilisin/kexin type 9 (PCSK9) binds to the EGF-like repeat (EGF-A) domain of low-density lipoprotein receptors (LDLR) and prevent LDLR recycling to the cell surface (CG Davis, et al., Acid-dependent ligand dissociation and recycling of LDL receptor mediated by growth factor homology region, Nature 1987; 326: 760-765). Using the non-competing method, PCSK9 blocking antibodies can be separated from non-blocking antibodies and can further enrich for the isolation of either blocking antibodies or non-blocking antibodies.

FIG. 8A-8C are representative flow staining profiles that demonstrate the detection and separation of the two different anti-PSCK9 antibody coated beads in two different non-competing method conditions (FIGS. 8A and 8B). Beads coated with antibody R007, a PCSK9 blocking antibody, are shown in FIG. 8A. 99.8% of the population appeared in Q1 quadrant as the A647-positive and PE-negative population, showing that R007 bound to PCSK9 (A647) and prevented PCSK9 further interacting with LDLR (PE). Beads coated with antibody R008, a PCSK9 non-blocking antibody, were shown to have 83.7% in the A647 and PE double positive gate (Q2 quadrant), with a 16.2% tail in the A647 only gate (Q1 quadrant) when biotin labeled LDLR and streptavidin tetramer PE were added sequentially (FIG. 8B), and beads coated with antibody R008 was shown to increase to 99.1% of the total population in the A647 and PE double positive gate (Q2 quadrant), with only a residual tail of 0.85% in the A647 only gate (Q1 quadrant) when biotin labeled LDLR and streptavidin tetramer PE were pre-clustered (FIG. 8C).

### DETAILED DESCRIPTION

Disclosed herein are methods for obtaining antibody-producing cells that express antibody molecules which bind to a targeted antigen without competing to one or more other binding partner(s). Also disclosed herein are methods for detecting and differentiating antibody-producing cells that express antibody molecules which bind to a targeted antigen without competing to one or more other binding partner(s), and antibody-producing cells that express antibody molecules which bind to a targeted antigen and block the binding by another binding partner. The methods disclosed herein enables enrichment or isolation of cells expressing target antigen-specific antibody molecules that are non-competing to one or more additional binding partners, or enrichment or isolation of cells expressing target antigen-specific antibody molecules that block binding by another additional binding partner.

The terms “aspect” and “embodiment” are used herein to describe the various feature(s) and characteristic(s) of the methods or method steps. The feature(s) and characteristic(s) described in one aspect or embodiment may be combined with those in one or more other aspects or embodiments in any appropriate manner.

Although claimed subject matter will be described in terms of certain examples, other examples, including examples that do not provide all the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

The term “about” as used herein means plus or minus 10%, or plus or minus 5%, or plus or minus 4%, or plus or minus 3%, or plus or minus 2%, or plus or minus 1%, from a specified value, as well as the specified value itself.

In the description that follows, certain conventions will be followed as regards to the usage of terminology. Generally, terms used herein are intended to be interpreted consistently with the meaning of those terms as they are known to those of skill in the art. In practicing the present disclosure, many conventional techniques in molecular biology, microbiology, cell biology, biochemistry, and immunology are used, which are within the skill of the art. These techniques are described in greater detail in, for example, Molecular Cloning: a Laboratory Manual 4th edition, J. F. Sambrook and D. W. Russell, ed. Cold Spring Harbor Laboratory Press 2012; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the disclosure.

### General Description

Disclosed herein are methods for detecting cells expressing antibody molecules that bind to a targeted antigen without competing with one or more additional binding partners of the target antigen. The target antigen can be a monomeric antigen or a multimeric antigen. Binding partners of a target antigen, also referred to as “binders” herein, can be an antibody or an antigen-binding fragment thereof, or a receptor or a ligand for the target antigen. Also disclosed herein are methods for detecting and differentiating cells expressing antibody molecules to a target antigen that are non-competing with one or more binding partners, from cells expressing antibody molecules to a target antigen that block binding by a binding partner. The methods disclosed herein allow for enrichment and isolation of cells expressing non-competing antibody molecules to a target antigen, and cells expressing blocking antibody molecules to a target antigen.

The term “antibody molecule”, as used herein, encompasses both full length antibodies and antigen-binding fragments thereof (e.g., Fab, Fab′, and F(ab′)₂).

The terms “compete”, “competing”, “non-competing” and “non-competitive”, as used herein, describe features of binding by multiple (two or more) antibody molecules to an antigen. Antibody molecules that do not compete, i.e., are non-competitive, for binding to an antigen are able to bind to the antigen simultaneously. Non-competitive antibody molecules bind to distinct epitopes of the antigen. On the other hand, antibody molecules that compete, i.e., are competitive, for binding to an antigen generally cannot bind to the antigen simultaneously.

Competitive antibody molecules may bind to the same epitope, overlapping epitopes, or to adjacent epitopes on the antigen. Binding to an antigen by an antibody can block subsequent binding by a competitive antibody.

The term “enriching” means increasing the frequency or percentage of desired cells in a cell population. Thus, an antibody-producing cell population enriched in cells expressing antibody molecules which do not compete with at least one additional binding partner for the antigen of interest encompasses an antibody-producing cell population having a higher frequency and/or higher percentage of antibody-producing cells expressing antibody molecules to an antigen of interest which do not compete with at least one additional binding partner for the antigen of interest as a result of the methods disclosed herein, as compared to a starting cell population without or before being subjected to the methods disclosed herein. An antibody-producing cell population enriched in cells expressing antibody molecules which block the binding by at least one additional binding partner for the antigen of interest encompasses an antibody-producing cell population having a higher frequency and/or higher percentage of antibody-producing cells expressing antibody molecules to an antigen of interest which block the binding by at least one additional binding partner for the antigen of interest as a result of the methods disclosed herein, as compared to a starting cell population without or before being subjected to the methods disclosed herein.

The extent of enrichment, i.e., the extent of increase of the percentage of desired cells in a cell population after performing the methods disclosed herein, may be presented by the percent or fold increase of desired cells, as compared to the desired cells after performing a conventional method. For example, the percentage of desired cells may be increased from 40% to 50%, resulting in an improvement or enrichment (i.e., an increase of desired cells by 25%). In some embodiments, the extent of enrichment may be at least 5% to 10%. In some embodiments, the extent of enrichment may be at least 10% to 15%. In some embodiments, the extent of enrichment may be at least 15% to 20%. In some embodiments, the extent of enrichment may be at least 20% to 25%. In some embodiments, the extent of enrichment may be at least 25% to 30%. In some embodiments, the extent of enrichment may be at least 30% or greater, e.g., 50% (0.5 fold) or 100% (1 fold) In some embodiments, the enrichment of the present methods is reflected by an increase of the percentage of desired cells in a cell population after performing the methods, as compared to the percentage of desired cells in a cell population after performing a conventional method, such as a direct sort process which is also described in this disclosure. In some embodiments, the extent of enrichment of desired cells (e.g., cells expressing antibody molecules to an antigen of interest which do not compete with at least one additional binding partner for the antigen of interest) based on comparison to a direct sort process is at least 10% or at least 15%. In some embodiments, the extent of enrichment of desired cells based on comparison to a direct sort process is at least 20% or at least 25%. In some embodiments, the extent of enrichment of desired cells based on comparison to a direct sort process is at least 30% or greater, e.g., at least 50% or 100%.

Alternatively, the level of enrichment, i.e., the level of increase of desired cells in a cell population after performing the methods disclosed herein, may be presented by the percent decrease of the undesired cells (e.g., cells expressing antibody molecules to an antigen of interest which compete with at least a binding partner for the antigen of interest) after performing a conventional method. In some embodiments, the level of enrichment of desired cells is reflected by at least 30% to 50% decrease of undesired cells. In some embodiments, the level of enrichment of desired cells is reflected by at least 50% to 75% decrease of undesired cells. In some embodiments, the level of enrichment of desired cells is reflected by at least 75% to 100% decrease of undesired cells. In some embodiments, the level of enrichment of desired cells is reflected by at least 200% (2 fold) decrease of undesired cells. In some embodiments, the level of enrichment of desired cells is reflected by at least 5 or 10 fold decrease of undesired cells. In some embodiments, the level of enrichment of desired cells is reflected by at least 20 or 30 fold decrease of undesired cells. In some embodiments, the enrichment of the present methods is reflected by a decrease of the percentage of undesired cells in a cell population after performing the methods, as compared to the percentage of undesired cells in a cell population after performing a conventional method, such as a direct sort process which is also described in this disclosure. In some embodiments, the level of enrichment of desired cells is reflected by a decrease of the percentage of undesired cells in a cell population after performing the methods, as compared to the percentage of undesired cells in a cell population after performing a direct sort process, wherein the decrease is at least 0.5 fold, at least 1 fold, at least 2 fold, at least 5 fold, at least 10 fold, or at least 20 fold.

The methods may be carried out in several different formats, which may depend on whether the antigen is a monomer or multimer.

Non-Compete Sorting with Monomeric, Unlabeled Target Antigen

In one aspect, disclosed herein is a method that utilizes a monomeric, unlabeled target antigen to sort antibody-producing cells in order to obtain cells expressing antibody molecules that are specific to the target antigen and are non-competitive with one or more binding partners (binders) of the target antigen.

In some embodiments, the method comprises (a) contacting a population of antibody-producing cells with: a target antigen, and a first binder to the target antigen, wherein the first binder is conjugated with a detectable label, to permit binding of cells that express antibody molecules that are non-competitive with the first binder to bind to the target antigen; and (b) collecting cells that are bound to the target antigen which is bound by the first binder conjugated to the detectable label, thereby obtaining a population of antibody-producing cells expressing antibody molecules that are non-competitive with the first binder.

In some embodiments, the target antigen is prebound with the first binder prior to being contacted by the population of antibody-producing cells.

In some embodiments, the target antigen is not prebound with the first binder, but is prebound with at least one other binder that does not compete with the first binder in binding to the target antigen, and the contacting of a population of antibody-producing cells with the target antigen (prebound with at least one other binder) and with the first binder permits cells that express antibody molecules that are non-competitive with the first binder and non-competitive with the at least one other binder to bind to the target antigen. In some embodiments, the at least one other binder is one other binder that is non-competitive with the first binder; and in other embodiments, the at least one other binder includes two or more binders that are non-competitive with the first binder. Cells subsequently collected that remain bound to the target antigen and to the first binder are cells which express antibody molecules that do not compete with the first binder and do not compete with any of the prebound binders.

In some embodiments, the target antigen is not prebound with any binder prior to being contacted by the population of antibody-producing cells.

By ‘prebound”, it means a molecule is bound with one or more of its binding partner prior to subsequent incubation steps. In some embodiments, the binding interaction between a molecule and its prebound binding partner is non-covalent. In some embodiments, the binding interaction between a molecule and its prebound binding partner is covalent. In the present methods, a target antigen can be prebound by a binder through incubating the target antigen with the binder for at least 30 minutes at room temperature or at least 45 minutes at 4° C. For this prebinding step, the binder is in excess (e.g., between 5 to 10 fold and up to 100 fold) relative to the concentration of the target antigen.

In embodiments where the target antigen is not prebound with the first binder prior to contacting the population of antibody-producing cells, irrespective of whether the target antigen is prebound with any other binder, the cells can be contacted by the target antigen first, and subsequently by the first binder; alternatively, the cells can be contacted by the target antigen and the first binder simultaneously. When the cells are contacted by the target antigen and the first binder sequentially, a wash step can be included between the contacting with the target antigen and the contacting with the first binder to remove any unbound target antigen. The wash step can be repeated if needed.

In referring to contacting the cells with a target antigen and/or with a first binder, it is meant that the cells are incubated in a solution containing the target antigen and/or the first binder. Generally speaking, a target antigen can be brought into contact with cells at a concentration from 0.02 nM to 25 nM, e.g., at 1 nM, 2 nM, 3 nM, 4 nM, 5 nM, 6 nM, 7 nM, 8 nM, 9 nM, or 10 nM. In general, a first binder can be brought into contact with the cells at a concentration from 0.01 nM to 100 nM, for example, a concentration from 0.1 nM to 50 nM, 0.5 nM to 25 nM, or 1 nM-10 nM. A first binder can be brought into contact with the cells at a concentration as low as one fifth of the KD between the first binder molecule and the target antigen. For example, if the first binder is an ultra high affinity antibody with KD at 1 pM, then it can be added at a concentration at 0.2 pM or higher. Alternatively, if the first binder is a receptor with relatively weak binding affinity to the target antigen (ligand) with KD at 100 nM, then it should be added at 20 nM or higher.

The “contacting” or incubation of the cells with a target antigen, and/or with a first binder, can be performed for at least several minutes, for example, at least 5 minutes. In some embodiments, the incubation can be performed for at least 10 minutes. In some embodiments, the incubation can be performed for at least 15 minutes. In some embodiments, the incubation can be performed for at least 30 minutes. In some embodiments, the incubation can be performed for at least 45 minutes. In some embodiments, the incubation can be performed for at least an hour. In some embodiments, the incubation can be performed for at least 2 hours. In some embodiments, the incubation can be performed for at least 3 hours. In some embodiments, the incubation can be performed for at least 4 hours. In some embodiments, the incubation can be performed for at least 5 hours. In some embodiments, the incubation can be performed for at least 6 hours. In some embodiments, the incubation can be performed for at least 12 hours. In some embodiments, the incubation can be performed for at least 15 hours. In some embodiments, the incubation can be performed for at least 18 hours. In some embodiments, the incubation can be performed for at least 24 hours. In some embodiments, the incubation is performed at room temperature (about 23-27° C.). In some embodiments, the contacting is performed at 4° C. When the incubation is performed at room temperature, the duration of the incubation is generally shorter than when the incubation is performed at 4° C. Suitable conditions for incubation include, e.g., 5-15 minutes or up to 30-45 minutes at room temperature, or several hours or overnight at 4° C.

After the cells have been contacted with the target antigen and the first binder (which is conjugated with a detectable label), with or without at least one other binder, cells that are bound to the target antigen which is in turn bound by the first binder conjugated to the detectable label and optionally bound by at least one other binder, can be collected by any suitable means, e.g., FACS. A wash step can be included prior to collecting the cells to remove any unbound components (e.g., unbound target antigen, unbound first binder, or unbound other binder(s)). The wash step can be repeated if needed. The collected cells are enriched in cells expressing antibody molecules that are specific to the target antigen and are non-competitive with the first binder.

Non-Compete Sorting with Multimeric, Labeled Target Antigen

In one aspect, disclosed herein is a method that utilizes a multimeric, labeled target antigen to sort antibody-producing cells to obtain cells expressing antibody molecules that are specific to the target antigen and are non-competitive with a binding partner (binder) of the target antigen.

In some embodiments, the method comprises (a) contacting a population of antibody-producing cells with a multimeric target antigen, wherein the target antigen is conjugated with a detectable label and is prebound by a first binder to permit binding of cells that express antibody molecules that are non-competitive with the first binder to bind to the target antigen; and (b) collecting cells that are bound to the target antigen that is conjugated with the detectable label.

In some embodiments, the target antigen is prebound by a first binder and at least one other binder, and the cells collected are cells that express antibody that does not compete with either the first binder or the at least one other binder. In such embodiments, the method comprises: (a) contacting a population of antibody-producing cells with a target antigen, wherein the target antigen is conjugated with a detectable label and is prebound by a first binder and at least one other binder, to permit binding of cells that express antibody molecules that are non-competitive with either the first binder or the at least one other binder in binding to the target antigen; and (b) collecting cells that are bound to the target antigen that is conjugated with the detectable label. In some embodiments, the at least one other binder includes two or more binders.

In some embodiments, the concentration of the first binder is in excess (e.g., 5-10 fold to 100 fold) relative to the concentration of the target antigen. In embodiments where the antigen is also prebound to at least one other binder, the at least one other binder is also in excess (e.g., 5-10 fold to 100 fold) relative to the concentration of the target antigen.

Generally speaking, a target antigen can be brought into contact with cells at a concentration from 0.02 nM to 25 nM, e.g., at 1 nM, 2 nM, 3 nM, 4 nM, 5 nM, 6 nM, 7 nM, 8 nM, 9 nM, or 10 nM.

The “contacting” or incubation of the cells with the target antigen prebound by the first binder and optionally at least one other binder can be performed for at least several minutes, for example, at least 5 minutes. In some embodiments, the incubation can be performed for at least 10 minutes. In some embodiments, the incubation can be performed for at least 15 minutes. In some embodiments, the incubation can be performed for at least 30 minutes. In some embodiments, the incubation can be performed for at least 45 minutes. In some embodiments, the incubation can be performed for at least an hour. In some embodiments, the incubation can be performed for at least 2 hours. In some embodiments, the incubation can be performed for at least 3 hours. In some embodiments, the incubation can be performed for at least 4 hours. In some embodiments, the incubation can be performed for at least 5 hours. In some embodiments, the incubation can be performed for at least 6 hours. In some embodiments, the incubation can be performed for at least 12 hours. In some embodiments, the incubation can be performed for at least 15 hours. In some embodiments, the incubation can be performed for at least 18 hours. In some embodiments, the incubation can be performed for at least 24 hours. In some embodiments, the incubation is performed at room temperature (about 23-27° C.). In some embodiments, the contacting is performed at 4° C. When the incubation is performed at room temperature, the duration of the incubation is generally shorter than when the incubation is performed at 4° C. Suitable conditions for incubation include, e.g., 5-15 minutes or up to 30-45 minutes at room temperature, or several hours or overnight at 4° C.

After the cells have been contacted with the target antigen and the first binder and optionally at least one other binder, cells that are bound to the target antigen which is bound by the first binder and optionally at least one other binder and is conjugated to the detectable label, can be collected. A wash step can be included prior to collecting the cells to remove any unbound components (unbound target antigen). The wash step can be repeated if needed. The collected cells are enriched in cells expressing antibody molecules that are specific to the target antigen and are non-competitive with the first binder, or in cases where at least one other binder is employed, are non-competitive with either the first binder or the at least one other binder.

Sorting with Monomeric, Labeled Target Antigen for Noncompetitive Antibody Molecules and/or Blocking Antibody Molecules

In a further aspect, disclosed herein is a method that utilizes a monomeric, labeled target antigen to sort antibody-producing cells to obtain cells expressing antibody molecules that are specific to the target antigen and are non-competitive with one or more binding partners of the target antigen, and/or cells expressing antibody molecules that are specific to the target antigen and block the binding by a binding partner to the target antigen.

In some embodiments, the method comprises (a) contacting a population of antibody-producing cells with a target antigen conjugated to a first detectable label, and a first binder to the target antigen, wherein the first binder is conjugated with a second detectable label that is different from the first detectable label, to permit binding of cells that express antibody molecules that are non-competitive with the first binder to bind to the target antigen; and (b) collecting cells that are bound to: (1) the target antigen conjugated to the first detectable label but not the first binder conjugated with the second detectable label, thereby obtaining a population of antibody-producing cells expressing antibody molecules that block the first binder in binding to the target antigen; and/or (2) the target antigen conjugated to the first detectable label and the first binder conjugated to the second detectable label thereby obtaining a population of antibody producing cells expressing antibody molecules that are non-competitive with the first binder in binding to the target antigen.

In some embodiments, for step (a), the target antigen conjugated to the first detectable label is prebound with the first binder conjugated to the second detectable label, prior to contacting the population of antibody-producing cells. Cells collected that remain bound to the target antigen and the first binder are cells which express antibody molecules that do not compete with the first binder. On the other hand, cells collected that remain bound to the target antigen but not to the first binder (which has been kicked off) are cells which express antibody molecules that compete with and replace the first binder in binding to the target antigen.

In some embodiments, the target antigen is not prebound with the first binder, rather, the target antigen is prebound with at least one other binder before contacting the population of antibody-producing cells, and wherein the at least one other binder does not compete with the first binder. In some embodiments, the at least one other binder comprises two or more binders that do not compete with the first binder. Cells collected that remain bound to the target antigen and to the first binder are cells which express antibody molecules that do not compete with any of the binders. On the other hand, cells collected that remain bound to the target antigen but not to the first binder are cells which express antibody molecules that do not compete with any of the binders prebound to the target antigen, yet block binding by the first binder.

In embodiments where the target antigen is not prebound with the first binder prior to contacting the population of antibody-producing cells, irrespective of whether the target antigen is prebound with any other binder, the cells can be contacted by the target antigen (conjugated to the first detectable label) first, and subsequently by the first binder (conjugated to the second detectable label); or alternatively, the cells can be contacted by the target antigen (conjugated to the first detectable label) and the first binder (conjugated to the second detectable label) at the same time. When the cells are contacted by the target antigen and the first binder sequentially, a wash step can be included between the contacting with the target antigen and the contacting with the first binder to remove any unbound target antigen. The wash step can be repeated if needed.

In some embodiments, the first binder, which can be an antibody or an antigen-binding fragment thereof, a receptor, or a ligand, can be provided in a multiple valent form. By “multivalent” it is meant multiple units of a molecule (e.g., a binder). A multivalent molecule can be a dimer, trimer, tetramer, pentamer, hexamer, and the like, or a combination thereof. In some embodiments, the multivalent molecule is a streptavidin multimer (e.g., tetramer), which can be complexed with biotin which is then linked to a binder, thereby providing a multivalent (e.g., tetravalent) form of the binder. In some embodiments, a streptavidin multimer includes tetramer and may additionally include trimer and/or dimer. In some embodiments, a streptavidin multimer is conjugated with a fluorophore such as phycoerythrin. In some embodiments, the multivalent molecule is a dimer of an immunoglobulin Fc fragment, to which a binder can be linked to provide a bivalent form of the binder. In some embodiments, the multivalent molecule is a trimer of a trimerization molecule, such as foldon, to which a binder can be linked to provide a trivalent form of the antigen.

In some embodiments, a multivalent form is provided by a multimer (e.g., streptavidin or foldon) pre-clustered with a first binder (such as a receptor); for example, streptavidin preclustered with a low-density lipoprotein receptor (LDLR) or the EGF-like repeat (EGF-A) domain of the LDLR.

Generally speaking, a target antigen can be brought into contact with cells at a concentration from 0.02 nM to 25 nM, e.g., at 1 nM, 2 nM, 3 nM, 4 nM, 5 nM, 6 nM, 7 nM, 8 nM, 9 nM, or 10 nM. In general, a first binder can be brought into contact with the cells at a concentration from 0.01 nM to 100 nM, for example, a concentration from 0.1 nM to 50 nM, 0.5 nM to 25 nM, or 1 nM-10 nM. A first binder can be brought into contact with the cells at a concentration as low as one fifth of the KD between the first binder molecule and the target antigen. For example, if the first binder is an ultra high affinity antibody with KD at 1 pM, then it can be added at a concentration at 0.2 pM or higher. On the other hand, if the first binder is a receptor with relatively weak binding affinity to the target antigen (ligand) with KD at 100 nM, then it should be added at 20 nM or higher.

The “contacting” or incubation of cells with a target antigen and/or with a first binder can be performed for at least several minutes, for example, at least 5 minutes. In some embodiments, the incubation can be performed for at least 10 minutes. In some embodiments, the incubation can be performed for at least 15 minutes. In some embodiments, the incubation can be performed for at least 30 minutes. In some embodiments, the incubation can be performed for at least 45 minutes. In some embodiments, the incubation can be performed for at least an hour. In some embodiments, the incubation can be performed for at least 2 hours. In some embodiments, the incubation can be performed for at least 3 hours. In some embodiments, the incubation can be performed for at least 4 hours. In some embodiments, the incubation can be performed for at least 5 hours. In some embodiments, the incubation can be performed for at least 6 hours. In some embodiments, the incubation can be performed for at least 12 hours. In some embodiments, the incubation can be performed for at least 15 hours. In some embodiments, the incubation can be performed for at least 18 hours. In some embodiments, the incubation can be performed for at least 24 hours. In some embodiments, the incubation is performed at room temperature (about 23-27° C.). In some embodiments, the contacting is performed at 4° C. When the incubation is performed at room temperature, the duration of the incubation is generally shorter than when the incubation is performed at 4° C. Suitable conditions for incubation include, e.g., 5-15 minutes or up to 30-45 minutes at room temperature, or several hours or overnight at 4° C.

After the cells have been contacted with the target antigen and the first binder, desirable cells can be collected by suitable means, e.g., FACS, including two dimensional FACS. A wash step can be included prior to collecting the cells to remove any unbound components (unbound target antigen). The wash step can be repeated if needed. The collected cells are enriched in cells either expressing antibody molecules that are specific to the target antigen and are non-competitive with the first binder, or antibodies that are specific to the target antigen and block the binding of the first binder, or both, depending on which population or populations of cells are collected.

### Antigens of Interest, or Target Antigen

The methods disclosed herein are available for use with any antigen of interest. An antigen is any substance that results in an immune response. In some embodiments, the antigen of interest is a soluble protein. In some embodiments, the antigen of interest is a transmembrane protein. The antigen of interest can be any substance to which an antibody can bind including, but not limited to, peptides, proteins or fragments thereof; carbohydrates; organic and inorganic molecules; receptors produced by animal cells, bacterial cells, and viruses; enzymes; agonists and antagonists of biological pathways; hormones; and cytokines. Exemplary antigens include, but are not limited to, IL-2, IL-4, IL-6, IL-10, IL-12, IL-13, IL-18, IFN-α, IFN-γ, Angiotensin II, BAFF, CGRP, CXCL13, IP-10, PCSK9, NGF, Nay1.7, VEGF, EPO, EGF, and HRG. In some embodiments, the antigen of interest is a cytokine such as an interleukin (IL). In some embodiments, the antigen of interest is a growth factor. In some embodiments, the antigen of interest may be a tumor marker or tumor associated antigen. In some embodiments, the antigen of interest is a surface protein from a pathogen (such as a virus or a bacterium). In some embodiments, the antigen of interest is a viral protein.

In some embodiments, the antigen is a protein that is present in a monomeric form. Examples of proteins that exist in monomers include, but are not limited to, interleukin molecules, a SARS-COV-2 RBD (receptor binding domain) Protein, an Ebola glycoprotein subunit, MERS-COV RBD monomer protein, Malaria sporozoite surface protein, and Proprotein convertase subtilisin/kexin type 9 (PCSK9). In some embodiments, the antigen is a protein that is present in a multimeric form, including homomers and heteromers. Examples of multimeric antigens include an Ebola glycoprotein trimer, an Influenza HA trimer protein, a SARS-COV-1 Spike protein, and a MERS-COV Spike protein.

### Binding Partner, or Binder

Binding partners, or “binders” of a target antigen, encompass any molecule that binds to a target antigen. For example, when the target antigen is a polypeptide, a binder may bind to several amino acids in the target antigen. A binder can be a protein, a nucleic acid, or another organic compound or polymer. A protein binder can be an antibody, or an antigen-binding fragment of an antibody, e.g., polypeptides containing the variable regions of heavy chains and light chains of an antibody (e.g., Fab fragment, F(ab′)₂ fragment). A protein binder can also be a receptor or a ligand-binding domain thereof, where the target antigen is the ligand for the receptor. A protein binder can also be a ligand, where the target antigen is the receptor for the ligand or a ligand-binding domain of the receptor. As an example, the target antigen is PCSK9, and the binder is a low-density lipoprotein receptor (LDLR) or the EGF-like repeat (EGF-A) domain of the LDLR.

### Detectable Labels

In various embodiments, a target antigen or a binder is conjugated to a detectable label. Detectable labels, as used herein, refer to molecules or compounds that provide, directly or indirectly, signals that can be detected. Detectable labels include small molecules, radioisotopes, enzymatic proteins and fluorescent dyes.

In some embodiments, the detectable label is a small molecule. Detectable small molecule labels allow for easy labeling of proteins and can be used in a number of regularly deployed detection assays known in the art.

In some embodiments, the detectable label is an enzyme reporter. Enzyme labels are larger than biotin, however, they rarely disrupt antibody function. Commonly used enzyme labels are horseradish peroxidase (HRP), alkaline phosphatase (AP), glucose oxidase and 0-galactosidase. To use enzyme-labeled antibody molecules, samples are incubated with an enzyme-specific substrate that is catalyzed by the enzyme to produce a colored product (chromogenic assays) or light (chemiluminescent assays). Each enzyme has a set of substrates and detection methods that can be employed. For example, HRP can be reacted with diaminobenzidine to produce a brown-colored product or with luminol to produce light. In contrast, AP can be reacted with para-Nitrophenylphosphate (pNPP) to produce a yellow-colored product detected by a spectrophotometer or with 5-bromo-4-chloro-3-indolyl phosphate (BCIP) and nitroblue tetrazolium (NBT) to produce a purple-colored precipitate.

In some embodiments, the detectable label is a fluorescent label. Fluorescent tags can be covalently attached to proteins through primary amines or thiol.

A target antigen or a binder can be conjugated (i.e., linked or bound) to a label which directly provides a signal that is detectable, or to a label which can be detected through interaction with, or action by, another molecule, leading to a signal that is detectable. In one example, a target antigen or a binder can be conjugated directly to a fluorescent compound, or alternatively, a target antigen or a binder can be conjugated to biotin, which can be subsequently bound by streptavidin that is labeled with a fluorescent compound—in both instances, the target antigen and the binder are conjugated to a detectable label, biotin. In another example, a target antigen or a binder can be conjugated to a substrate of an enzyme, which generates a detectable signal when the enzyme is supplied—in this instance, the target antigen and the binder are conjugated to a detectable label, the substrate.

### Antibody-Producing Cells

The terms “antibody-producing cells” and “antibody-expressing cells” refer to cells that express antibody molecules on the cell surface, i.e., the antibody molecules are bound to or anchored in the cell membrane. Cell surface expression of antibody molecules can occur either naturally, e.g., as a result of B-cell activation, or as a result of recombinant technology and genetic engineering. The term, therefore, encompasses lymphocytes of antigen-dependent B-cell lineage, including memory B-cells, recombinant cells such as non-lymphoid cells engineered to express antibody molecules on the cell surface, including yeast and mammalian cells such as immortalized cells and hybridoma cells. In some embodiments, the immortalized cells include Chinese hamster ovary (CHO) cells, human embryonic kidney (HEK)293 cells, and murine myeloma cells (e.g., NS0 and Sp2/0).

In some embodiments, antibody-producing cells are primary antibody producing cells. “Primary cells” refers to cells isolated or harvested from living tissue or organ, e.g., cells isolated from a mammal. Primary cells can be cultured in vitro outside of the natural environment from which they are isolated. In certain embodiments, primary antibody-producing cells are tissue-derived, such as from spleen, lymph node, bone marrow, or peripheral blood. In some embodiments, antibody-producing cells may be derived from primary antibody-producing cells. For example, primary antibody-producing cells may be fused to myeloma cells to make hybridomas, or otherwise immortalized, such as infected with a virus (e.g., EBV), or may be differentiated by cell sorting techniques based on protein markers expressed by particular B cell types.

In some embodiments, antibody-producing cells are mammalian cells or yeast engineered to express antibody molecules on the cell surface. In the context of cells engineered to express antibody molecules, the cells may be engineered to express full length immunoglobulin molecules or antigen-binding fragments. In some embodiments, antibody-producing cells are yeast cells (e.g., S. cerevisiae or Pichia) engineered to express antibody molecules on the cell surface. In some embodiments, antibody-producing cells are mammalian cells engineered to express antibody molecules on the cell surface. In some embodiments, the mammalian cells are immortalized cells engineered to express antibody molecules on the cell surface, which include, e.g., CHO cells, HEK293 cells, and murine myeloma cells (e.g., NS0 and Sp2/0).

The noncompete sorting methodology described herein also applies to cell surface display platforms of various host cells, including yeast or mammalian cells such as CHO cells, that express antibody molecules on the cells surface to permit screening from antibody gene libraries or repertoires of antibody maturation variants.

Yeast surface display (YSD) platforms have been described and widely used in antibody screening (Border and Wittrup, Nat Biotechnol. 1997; 15:553-7; Feldhaus M J et al., Nat Biotechnol. 2003; 21:163-70; McMahon C et al., Nat Struct Mol Biol. 2018; 25:289-96). Display of Fab regions on the yeast surface has been reported to increase the antibody diversity and enlarge the library size (Weaver-Feldhaus J M et al., FEBS Lett. 2004; 564: 24-34; Rosowski S et al., Microb Cell Fact. 2018; 17:3; Sivelle C et al., MAbs. 2018; 10:720-9).

Mammalian cell surface display platforms that display full-length antibodies or Fab fragments on the surface of mammalian cells, including CHO cells, have been described, e.g., by Zhou et al. MAbs. 2010; 2(5): 508-518; Nguyen et al, Protein Engineering, Design & Selection, 2018, vol. 31 no. 3, pp. 91-101). In addition, suitable for use herein are mammalian cells that carry a single antibody gene can be transfected with a gene encoding activation induced deaminase (AID) which initiates somatic hypermutation (SHM) by converting deoxycytidines (dC) to deoxyuracils (dU), to mutate the antibody gene in cells during cell proliferation in the cell culture. See, e.g., Chen C. et al., Biotechnol Bioeng. 113, 39-51 (2016).

### Immunization and Collection of Primary Antibody-Producing Cells

Immunization of mammals including human and nonhuman animals can be done by any methods known in the art (see, for example, E. Harlow and D. Lane—Antibodies A Laboratory Manual, Cold Spring Harbor (1988); Malik and Lillehoj, Antibody techniques: Academic Press, 1994, CA). The antigen of interest is administered as a protein, protein fragment, protein-fusion, or DNA plasmid that contains the antigen gene of interest and expressing the antigen of interest using the host cellular expression machinery to express the antigen polypeptide in vivo. It is understood that the immunized mammal may be a human having been exposed to antigen and expressing humoral immunity for the antigen of interest. Antigen may be administered directly to a mammal, without adjuvant, or with adjuvant to aid in stimulation of the immune response. Adjuvants known in the art include, but are not limited to, complete and incomplete Freund's adjuvant, MPL+TDM adjuvant system (Sigma), or RIBI (muramyl dipeptides) (see O'Hagan, Vaccine Adjuvant, by Human Press, 2000, NJ). Without relying on a particular theory, adjuvant can prevent rapid dispersal of polypeptide by sequestering the antigen in a local depot and may contain factors that can stimulate host immune response.

Once an appropriate immune response has been achieved, antibody-producing cells are collected from the immunized animal.

Antibody-producing cells can be collected from different sources of an immunized animal, including but not limited to the spleen, lymph node, bone marrow, and peripheral blood. In some embodiments, following immunization, splenocytes are harvested from an immunized animal. In some embodiments, peripheral blood mononuclear cells (PBMCs) are harvested from an immunized animal.

In some embodiments of the methods, a population of antibody-producing cells are antibody-producing B cells. In some embodiments, antibody-producing B cells can be obtained from immunized animals and isolated by FACS based on cell-surface B cell markers. B cell markers are known in the art. For example, applicable B cell markers that can be detected through the use of FACS include, but are not limited to, IgG, IgM, IgE, IgA, IgD, CD1, CD5, CD19, CD20, CD21, CD22, CD23, CD24, CD25, CD27, CD30, CD38, CD40, CD78, CD80, CD138, CD319, TLR4, IL-6, PDL-2, CXCR3, CXCR4, CXCR5, CXCR6, IL-10, and TGFβ.

In some embodiments, following immunization, splenocytes are harvested from an immunized animal. Following removal of red blood cells by lysis, IgG+ antigen-positive B cells can be isolated and used as a primary antibody producing cell population in the method.

In some embodiments, peripheral blood mononuclear cells (PBMCs) are harvested from an immunized animal known to have humoral immunity to an antigen of interest. IgG+, antigen-positive B cells can then be isolated for use as primary antibody producing cells in the present method.

Collected primary antibody-producing cells, such as antibody-producing B cells, can be processed to enrich for cells that express antibody on the cell surface that is directed to an antigen of interest. In some embodiments, an initial step of purification is optionally performed to enrich for primary antibody-producing cells. Such purification includes affinity chromatography, also referred to as affinity purification. There are several types of affinity purification known in the art, such as ammonium sulfate precipitation, affinity purification with immobilized protein A, G, A/G, or L; and affinity purification with immobilized antigen.

### Washing

As is known in the art, washing is a technique where a wash buffer is used to remove unwanted components, for example, unbound target antigen, or unbound first binder. The unbound components can be removed in a non-limiting example by adding a wash buffer to the mixture of cells and the components (the target antigen and/or a first binder), centrifuging the mixture, and removing the supernatant which comprises wash buffer and unbound components.

Wash buffers are known in the art. Wash buffers and wash steps are used to remove unbound and excessive components. Wash buffers can be designed for specific techniques, such as ELISA, immunoblotting, or immunohistochemistry. Some wash buffers are compatible with many different immunoassays. In some embodiments, the wash buffer is a phosphate buffered saline (PBS) based wash buffer. In some embodiments, the wash buffer is a Tris buffered saline (TBS) wash buffer. In some embodiments, the wash buffer comprises a detergent. In some embodiments, the detergent is Tween-20.

The cells are washed with wash buffer for an allotted time in order to remove unbound components. This allotted amount of time will be an amount of time sufficient to remove unbound components. In some embodiments, this allotted time can be from about 5 minutes to about 60 minutes; multiple washes that total from 5 to 60 minutes may be used, e.g., 3 washes of 10 minutes or one 30-minute wash; 2-4 washes of 5-15 minutes each, etc. In one embodiment, washing the cells for a period of time comprising one (1) wash about 5 minutes, or about 10 minutes, or about 15 minutes, or about 20 minutes, or about 25 minutes, or about 30 minutes, or about 35 minutes, or about 40 minutes, or about 45 minutes, or about 50 minutes, or about 55 minutes, or about 60 minutes total, may be used. In some embodiments, washing the cells for a period of time comprising two (2) washes about 5 minutes each, or about 10 minutes each, or about 15 minutes each, or about 20 minutes each, or about 25 minutes each, or about 30 minutes each per wash, may be used. Additional washing intervals are contemplated, essentially equivalent to those described herein.

After washing and aspirating the supernatant comprising the wash buffer and unbound components, the pellet comprising cells bound with the expected components (a target antigen and/or a first binder) can be used in subsequent steps. In some embodiments, the cell pellet can be resuspended in a buffer and used in subsequent steps. In some embodiments, the buffer used to resuspend the pellet can be the same wash buffer. In some embodiments, the buffer used to resuspend the pellet can be a different buffer than the wash buffer.

### Collecting Cells, Fluorescence-Activated Cell Sorting (FACS)

In various embodiments, cells that remain bound to a target antigen and a first binder, or cells that remain bound to the target antigen only without the first binder, can be collected based on the detectable label on target antigen and/or on the first binder. In some embodiments, flow cytometry is used for collecting a desired cell population.

Flow cytometry is a popular analytical cell-biology technique that utilizes light to count and profile cells in a heterogenous fluid mixture. Flow cytometry is a particularly powerful method because it allows a researcher to rapidly, accurately, and simply collect data related to many parameters from a heterogeneous fluid mixture containing live cells. Fluorescence-Activated Cell Sorting (FACS) is a derivative of flow cytometry that adds an exceptional degree of functionality. Using FACS a researcher can physically sort a heterogeneous mixture of cells into different populations.

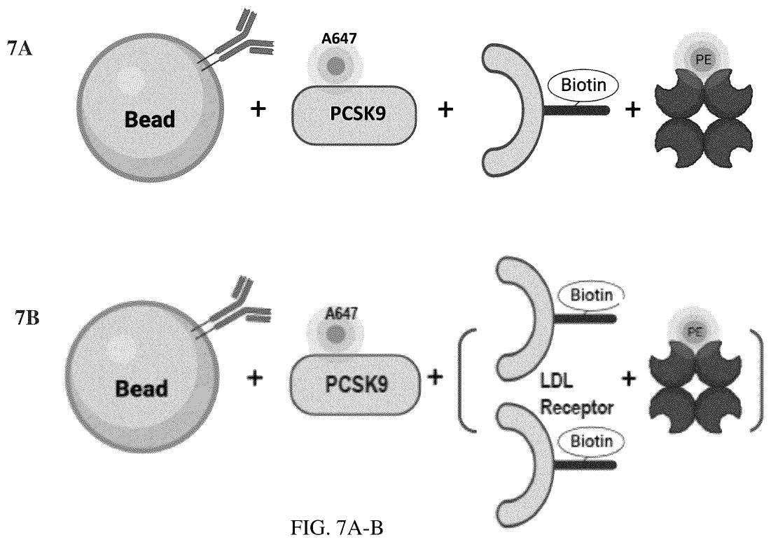

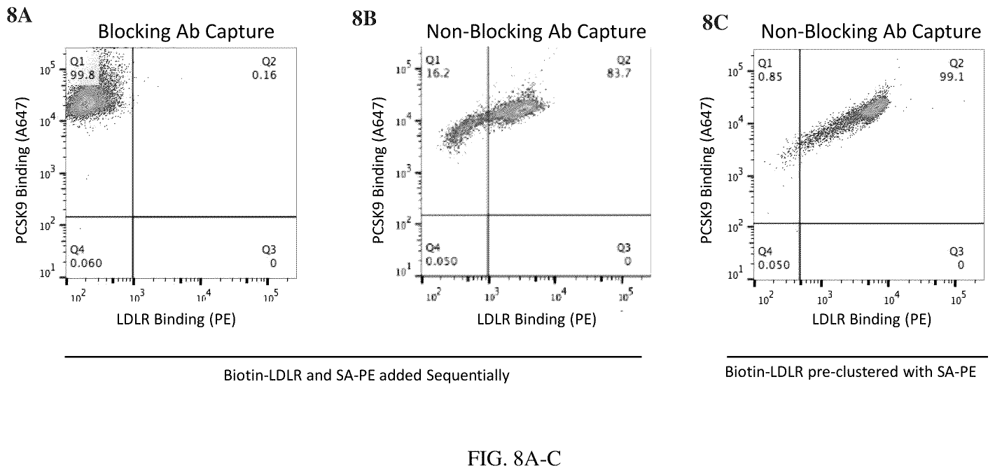

Two-dimensional (2D) FACS analysis is used in the sorting of cells based on two different fluorescent labels. Two-dimensional FACS analysis provides more selective results than FACS based on one parameter. 2D FACS analysis is used in some embodiments herein, e.g., to sort for cells expressing blocking antibody molecules (which only bind to the target antigen labeled with one fluorescent color) or alternatively cells expressing non-competing antibody molecules (that bind both the target antigen which in turn is bound by a binder labeled with a second fluorescent color), as depicted in FIGS. 7A-7B and 8A-8C. In some embodiments where the first detectable label is a first fluorescent label, and the second detectable label is a second fluorescent label that differentiates from the first fluorescent label, two-dimensional FACS analysis is used to collect cells that remain bound to the first label only, and/or cells that remain bound to both the first and second labels. For example, antibody-producing cells are contacted with a monomeric target antigen conjugated with a first detectable label, and with a first binder conjugated with a second detectable label; and after washing, cells that remain bound to only the first label are enriched in cells expressing antibody molecules that compete with and block the first binder in binding to the target antigen, and cells that remain bound to both the first label and the second label are enriched in cells expressing antibody molecules that are non-competitive with the first binder in binding to the target antigen. In specific embodiments, the first detectable label is A647 and the second detectable label is Phycoerythrin, or vice versa. It should be noted that the present methods are not limited by utilizing fluorescent labels, even in embodiments where 2D FACS analysis is employed. There may be additional fluorescent labels used for other purposes, e.g., use of a fluorescent label conjugated to an antibody specific for a B cell marker to gate for B cells. In other embodiments, two-dimensional (2D) FACS analysis can be used to separate and differentiate subsets of cells within cells that are collected. For example, to sort for cells expressing antibody that does not compete with a first binder and/or one other binder for a target antigen, the cells can be contacted with the target antigen, the first binder, and the other binder, wherein the target antigen is contacted with the antibody-expressing cells and subsequently with the first binder and/or the other binder, and wherein the first binder and the other binder are conjugated with two fluorescent labels which may be the same or different. In embodiments where the two fluorescent labels are different, 2D FACS analysis can be utilized to sort cells that express antibody that does not compete with either the first binder and/or with the other binder. Cells that remain bound only to the first binder (gated by the fluorescent label conjugated to the first binder) but not the other binder (gated by the fluorescent label conjugated to the other binder) are cells expressing antibody that does not compete with the first binder but does compete with the other binder; cells that remain bound only to the other binder but not the first binder are cells expressing antibody that competes with the first binder but does not compete with the other binder; and cells that remain bound to both binders are cells expressing antibody that does not compete with either binder.

In some embodiments, fluorescence-activated cell sorting (FACS) is used to select and sort single antibody-producing cells. Protocols for single cell isolation by flow cytometry are well-known (Huang, J. et al, 2013, supra). Single antibody-producing cells may be sorted and collected by alternative methods known in the art, including but not limited to manual single cell picking, limited dilution, B cell panning of adsorbed antigen, microfluidics, laser capture microdissection, and Gel Bead Emulsions (GEMs), which are all well-known in the art. See, for example, Rolink et al., J Exp Med (1996)183:187-194; Lightwood, D. et al, J. Immunol. Methods (2006) 316(1-2):133-43; Gross et al., Int. J. Mol. Sci. (2015) 16: 16897-16919; and Zheng et al., Nature Communications (2017) 8: 14049. Gel Bead Emulsions (GEMs) are also commercially available (e.g., 10× Chromium System from 10× Genomics, Pleasanton, CA).

Once collected, single antibody-producing cells may be propagated by common cell culture techniques for subsequent DNA preparation. Alternatively, antibody genes may be amplified from single antibody-producing cells directly and subsequently cloned into DNA vectors.

Generating Antibodies from Nucleic Acids Obtained from Antibody-Producing Cells

Nucleic acids encoding an antibody, or a fragment thereof, can be isolated from the antibody-producing cells obtained using the methods described herein.

In some embodiments, genes or nucleic acids encoding immunoglobulin variable heavy and variable light chains (i.e., VH and VL, and VL can be Vκ or Vλ chain) can be recovered using RT-PCR protocols with nucleic acids isolated from antibody-producing cells. These RT-PCR protocols are well known and conventional techniques, as described for example, by Wang et al., J. Immunol. Methods (2000) 244:217-225 and described herein.

In some embodiments, the nucleic acid encodes a fragment of an antibody, such as a variable domain, constant domain or combination thereof. In certain embodiments, the nucleic acid isolated from an antibody-producing cell encodes a variable domain of an antibody. In some embodiments, the nucleic acid encodes an antibody heavy chain or a fragment thereof (e.g., the variable domain of the antibody heavy chain). In other embodiments, the nucleic acid encodes an antibody light chain or a fragment thereof (e.g., the variable domain of the antibody light chain).

Once recovered, antibody-encoding genes or nucleic acids can be cloned into IgG heavy- and light-chain expression vectors and expressed via transfection of host cells. For example, antibody-encoding genes or nucleic acids can be inserted into a replicable vector for further cloning (amplification of the DNA) or for expression (stably or transiently) in cells. Many vectors, particularly expression vectors, are available or can be engineered to comprise appropriate regulatory elements required to modulate expression of an antibody encoding gene or nucleic acid.

An expression vector in the context of the present disclosure can be any suitable vector, including chromosomal, non-chromosomal, and synthetic nucleic acid vectors (a nucleic acid sequence comprising a suitable set of expression control elements) as described herein. Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In some embodiments, a nucleic acid molecule is included in a naked DNA or RNA vector, including, for example, a linear expression element (as described in, for instance, Sykes and Johnston, Nat Biotech (1997) 12:355-59), a compacted nucleic acid vector (as described in for instance U.S. Pat. No. 6,077,835), or a plasmid vector such as pBR322 or pUC 19/18. Such nucleic acid vectors and the usage thereof are well known in the art. See, for example, U.S. Pat. Nos. 5,589,466 and 5,973,972. In certain embodiments, the expression vector can be a vector suitable for expression in a yeast system. Any vector suitable for expression in a yeast system may be employed. Suitable vectors include, for example, vectors comprising constitutive or inducible promoters such as yeast alpha factor, alcohol oxidase and PGH. See, F. Ausubel et al., ed. Current Protocols in Molecular Biology, Greene Publishing and Wiley InterScience New York (1987); and Grant et al., Methods in Enzymol 153, 516-544 (1987).

In certain embodiments, the vector comprises a nucleic acid molecule (or gene) encoding a heavy chain of the antibody and a nucleic acid molecule (or gene) encoding a light chain of the antibody, wherein the antibody is produced by an antibody-producing cell that has been obtained by a method of the present disclosure.

Host cells suitable for expression of antibody molecules include, but are not limited to, cells of either prokaryotic or eukaryotic (generally mammalian) origin. In some embodiments, the host cell is a bacterial or yeast cell. In some embodiments, the host cell is a mammalian cell. In other embodiments, the host cell can be, for example, a Chinese hamster ovarian cells (CHO) such as, CHO K1, DXB-11 CHO, Veggie-CHO cells; a COS (e.g., COS-7); a stem cell; retinal cells; a Vero cell; a CV1cell; a kidney cell such as, for example, a HEK293, a 293 EBNA, an MSR 293, an MDCK, aHaK, a BHK21 cell; a HeLa cell; a HepG2 cell; WI38; MRC 5; Colo25; HB 8065; HL-60; a Jurkat or Daudi cell; an A431 (epidermal) cell; a CV-1, U937, 3T3 or L-cell; a C127 cell, SP2/0, NS-0 or MMT cell, a tumor cell, and a cell line derived from any of the aforementioned cells. In a particular embodiment, the host cell is a CHO cell. In a specific embodiment, the host cell is a CHO K1 cell.

It will be appreciated that the full-length antibody nucleic acid sequence or gene may be subsequently cloned into an appropriate vector or vectors. Alternatively, the Fab region of an isolated antibody may be cloned into a vector or vectors in line with constant regions of any isotype. Therefore, any constant region may be utilized in the construction of isolated antibodies, including IgG1, IgG2, IgG3, IgG4, IgM, IgA, IgD, and IgE heavy chain constant regions, or chimeric heavy chain constant regions. Such constant regions can be obtained from any human or animal species depending on the intended use of the antibodies. Also, antibody variable regions or Fab region may be cloned in an appropriate vector(s) for the expression of the protein in other formats, such as ScFv, diabody, etc.

In some embodiments, host cells comprising one or more of antibody-encoding nucleic acids are cultured under conditions that express a full-length antibody, and the antibody can then be produced and isolated for further use. In certain embodiments, the host cell comprises a nucleic acid that encodes a variable domain of an antibody, and the cell is cultured under conditions that express the variable domain. In other embodiments, the host cell comprises a nucleic acid that encodes a variable heavy chain (VH) domain of an antibody, and the cell is cultured under conditions that express the VH domain. In another embodiment, the host cell comprises a nucleic acid that encodes a variable light chain (VL) domain of an antibody, and the cell is cultured under conditions that express the VL domain. In specific embodiments, the host cell comprises a nucleic acid that encodes a VH domain of an antibody and nucleic acid that encodes a VL domain of an antibody, and the cell is cultured under conditions that express the VH domain and the VL domain.

### Exemplary Embodiments

In some embodiments, disclosed herein is a method for obtaining antibody-producing cells that express antibody molecules to a monomeric target antigen that are non-competitive with a first binder to the target antigen, wherein the first binder is an antibody or antigen binding fragment thereof, the method comprising:
(a) contacting a population of antibody-producing cells with:
   (1) the target antigen; and
   (2) the first binder to the target antigen, wherein the first binder is conjugated with a detectable label;
   to permit binding of cells that express antibody molecules that are non-competitive with the first binder to bind to the target antigen; and
(b) collecting cells that are bound to the target antigen which is bound by the first binder conjugated to the detectable label, thereby obtaining a population of antibody-producing cells expressing antibody molecules that are non-competitive with the first binder.

In some embodiments, disclosed herein is a method for obtaining antibody-producing cells that express antibody molecules to a monomeric target antigen that are non-competitive with a first binder to the target antigen, wherein the target antigen is a ligand and the first binder is a receptor for the ligand or a ligand-binding domain of the receptor, the method comprising:
(a) contacting a population of antibody-producing cells with:
   (1) the target antigen; and
   (2) the first binder to the target antigen, wherein the first binder is conjugated with a detectable label;
   to permit binding of cells that express antibody molecules that are non-competitive with the first binder to bind to the target antigen; and
(b) collecting cells that are bound to the target antigen which is bound by the first binder conjugated to the detectable label, thereby obtaining a population of antibody-producing cells expressing antibody molecules that are non-competitive with the first binder.

In some embodiments, disclosed herein is a method for obtaining antibody-producing cells that express antibody molecules to a monomeric target antigen that are non-competitive with a first binder to the target antigen, wherein the first binder is a ligand and the target antigen is a receptor for the ligand or a ligand binding domain of the receptor, the method comprising:
(a) contacting a population of antibody-producing cells with:
   (1) the target antigen; and
   (2) the first binder to the target antigen, wherein the first binder is conjugated with a detectable label;
   to permit binding of cells that express antibody molecules that are non-competitive with the first binder to bind to the target antigen; and
(b) collecting cells that are bound to the target antigen which is bound by the first binder conjugated to the detectable label, thereby obtaining a population of antibody-producing cells expressing antibody molecules that are non-competitive with the first binder.

In some embodiments, disclosed herein is a method for obtaining antibody-producing cells that express antibody molecules to a multimeric target antigen that are non-competitive with a first binder to the multimeric target antigen, wherein the first binder is an antibody or antigen binding fragment thereof, and wherein the multimeric target antigen is a multimeric protein (such as a glycoprotein of a virus), the method comprising:
(a) contacting a population of antibody-producing cells with the target antigen, wherein the target antigen is conjugated with a detectable label and is prebound by the first binder to permit binding of cells that express antibody molecules that are non-competitive with the first binder to bind to the target antigen; and
(b) collecting cells that are bound to the target antigen that is conjugated with the detectable label.

In some embodiments, disclosed herein is a method for obtaining antibody-producing cells that express antibody molecules to a multimeric target antigen that are non-competitive with a first binder to the multimeric target antigen, wherein the target antigen is a ligand and the first binder is a receptor for the ligand or a ligand-binding domain of the receptor, the method comprising:
(a) contacting a population of antibody-producing cells with the target antigen, wherein the target antigen is conjugated with a detectable label and is prebound by the first binder to permit binding of cells that express antibody molecules that are non-competitive with the first binder to bind to the target antigen; and
(b) collecting cells that are bound to the target antigen that is conjugated with the detectable label.

In some embodiments, disclosed herein is a method for obtaining antibody-producing cells that express antibody molecules to a multimeric target antigen that are non-competitive with a first binder to the multimeric target antigen, wherein the first binder is a ligand and the target antigen is a receptor for the ligand or a ligand binding domain of the receptor, the method comprising:
(a) contacting a population of antibody-producing cells with the target antigen, wherein the target antigen is conjugated with a detectable label and is prebound by the first binder to permit binding of cells that express antibody molecules that are non-competitive with the first binder to bind to the target antigen; and
(b) collecting cells that are bound to the target antigen that is conjugated with the detectable label.

In some embodiments, disclosed herein is a method for obtaining antibody-producing cells that express antibody molecules to a monomeric target antigen that are competitive and/or non-competitive with a first binder to the target antigen, wherein the first binder is an antibody or antigen binding fragment thereof, the method comprising:
(a) contacting a population of antibody-producing cells with:
   (1) the target antigen conjugated to a first detectable label; and
   (2) the first binder to the target antigen, wherein the first binder is conjugated with a second detectable label that is different from the first detectable label; to permit binding of cells that express antibody molecules that are non-competitive with the first binder to bind to the target antigen; and
(b) collecting cells that are bound to:
   (1) the target antigen conjugated to the first detectable label but not the first binder conjugated with the second detectable label, thereby obtaining a population of antibody-producing cells expressing antibody molecules that compete with the first binder in binding to the target antigen; and/or
   (2) the target antigen conjugated to the first detectable label and the first binder conjugated to the second detectable label thereby obtaining a population of antibody producing cells expressing antibody molecules that are non-competitive with the first binder in binding to the target antigen.

In some embodiments, disclosed herein is a method for obtaining antibody-producing cells that express antibody molecules to a monomeric target antigen that are competitive and/or non-competitive with a first binder to the target antigen, wherein the target antigen is a ligand and the first binder is a receptor for the ligand or a ligand-binding domain of the receptor, the method comprising:
(a) contacting a population of antibody-producing cells with:
   (1) the target antigen conjugated to a first detectable label; and
   (2) the first binder to the target antigen, wherein the first binder is conjugated with a second detectable label that is different from the first detectable label; to permit binding of cells that express antibody molecules that are non-competitive with the first binder to bind to the target antigen; and
(b) collecting cells that are bound to:
   (1) the target antigen conjugated to the first detectable label but not the first binder conjugated with the second detectable label, thereby obtaining a population of antibody-producing cells expressing antibody molecules that compete with the first binder in binding to the target antigen; and/or
   (2) the target antigen conjugated to the first detectable label and the first binder conjugated to the second detectable label thereby obtaining a population of antibody producing cells expressing antibody molecules that are non-competitive with the first binder in binding to the target antigen.

In some embodiments, disclosed herein is a method for obtaining antibody-producing cells that express antibody molecules to a monomeric target antigen that are competitive and/or non-competitive with a first binder to the target antigen, wherein the first binder is a ligand and the target antigen is a receptor for the ligand or a ligand binding domain of the receptor, the method comprising:
(a) contacting a population of antibody-producing cells with:
   (1) the target antigen conjugated to a first detectable label; and
   (2) the first binder to the target antigen, wherein the first binder is conjugated with a second detectable label that is different from the first detectable label; to permit binding of cells that express antibody molecules that are non-competitive with the first binder to bind to the target antigen; and
(b) collecting cells that are bound to:
   (1) the target antigen conjugated to the first detectable label but not the first binder conjugated with the second detectable label, thereby obtaining a population of antibody-producing cells expressing antibody molecules that compete with the first binder in binding to the target antigen; and/or
   (2) the target antigen conjugated to the first detectable label and the first binder conjugated to the second detectable label thereby obtaining a population of antibody producing cells expressing antibody molecules that are non-competitive with the first binder in binding to the target antigen.

### EXAMPLES

### Example 1. Sorting Non-Competing Antibodies with Monomeric Antigen

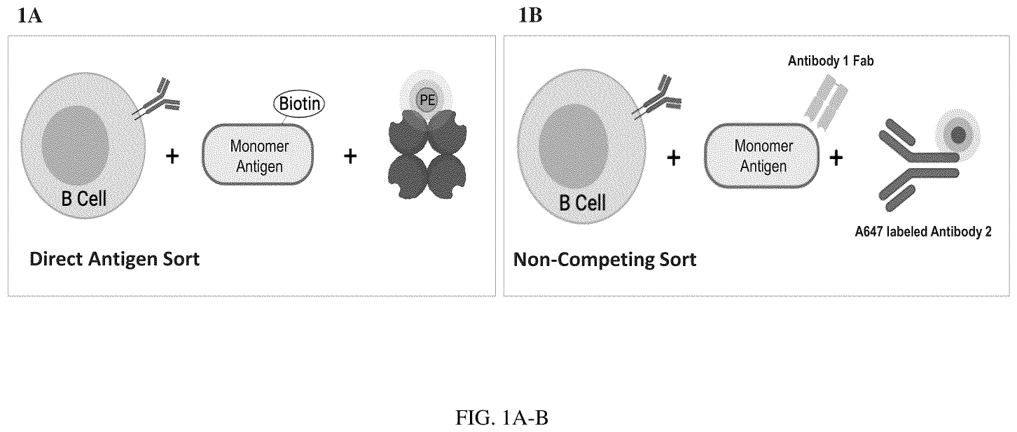

FIG. 1A demonstrates an exemplary embodiment of a direct antigen sort method for obtaining cells expressing antibody specific to a monomeric antigen. In this embodiment, antibody-producing cells (such as B cells) are contacted with a biotin labeled monomeric antigen. After removing unbound biotin labeled monomer, cells that are bound to the biotin labeled monomer can further be contacted with a fluorophore (e.g., Phycoerythrin (PE)) labeled streptavidin tetramer that forms complex with the biotin conjugated to the monomer bound to the cells. Cells labeled with the fluorophore can be collected and are cells that express antibody specific to the monomeric antigen.

In an alternative embodiment, antibody-producing cells can be contacted with a fluorophore conjugated monomeric protein (e.g., Phycoerythrin (PE) conjugated monomeric antigen) directly and cells labeled with the fluorophore can be collected and these are cells that express antibody specific to the monomeric antigen.

FIG. 1B demonstrates an exemplary embodiment of a non-competing sort method for obtaining cells expressing antibody that is specific to a monomeric antigen and does not compete with one or more antibodies (such as two antibodies as shown). In this embodiment, antibody producing cells (such as B cells) are contacted with an unlabeled monomer that is pre-bound to unlabeled Fab of Antibody 1. After removing the unbound monomer, the antibody-producing cells bound to the unlabeled monomer which is pre-bound with unlabeled Fab of Antibody 1 are further contacted with fluorophore (e.g., Alexa fluor 647) labeled Antibody 2 in the presence of excessive unlabeled Fab of Antibody 1. Antibody 1 and Antibody 2 do not compete with each other in binding to the monomer. Cells are then washed further in phosphate buffered saline (PBS) to remove unbound complex. Cells labeled with the fluorophore can be collected and these are cells that express antibody that is specific to the monomeric antigen and does not compete with Antibody 1 or Antibody 2.

Antibody-producing cells presenting antibodies bound to the monomer in both methods (FIGS. 1A and 1B) can be detected and isolated from other antibody-producing cells in a population using high-throughput techniques for single-cell isolation, such as fluorescence-activated cell sorting (FACS).

The direct antigen sort method depicted in FIG. 1A and the non-compete sort method depicted in FIG. 1B were applied to sort for B cells specific to the monomeric protein, SARS-CoV-2 RBD (i.e., the receptor-binding domain in the S1 subunit of the viral spike protein), as follows.

Spleens from SARS-CoV-2 RBD immunized mice were harvested, splenocytes were obtained, and red blood cells were removed by lysis. The cells were then stained with fluorochrome conjugated antibodies specific to a B cell surfaces marker, such as IgG, to identify B cells.

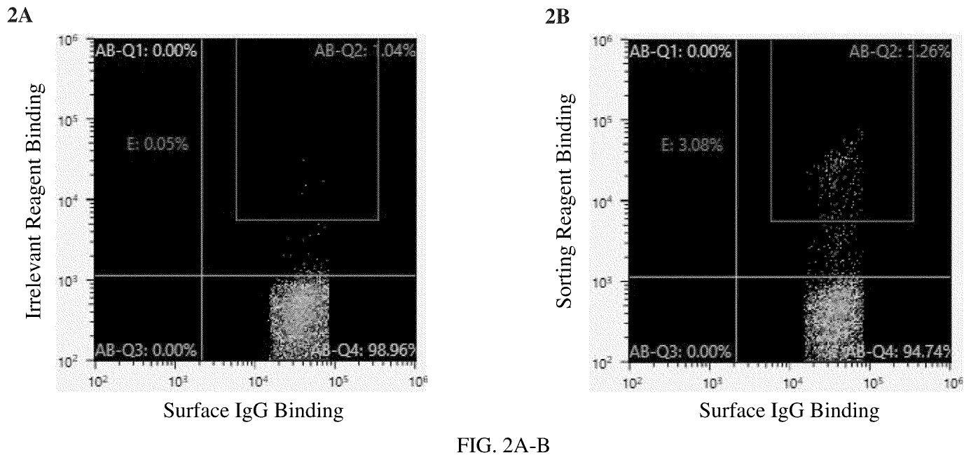

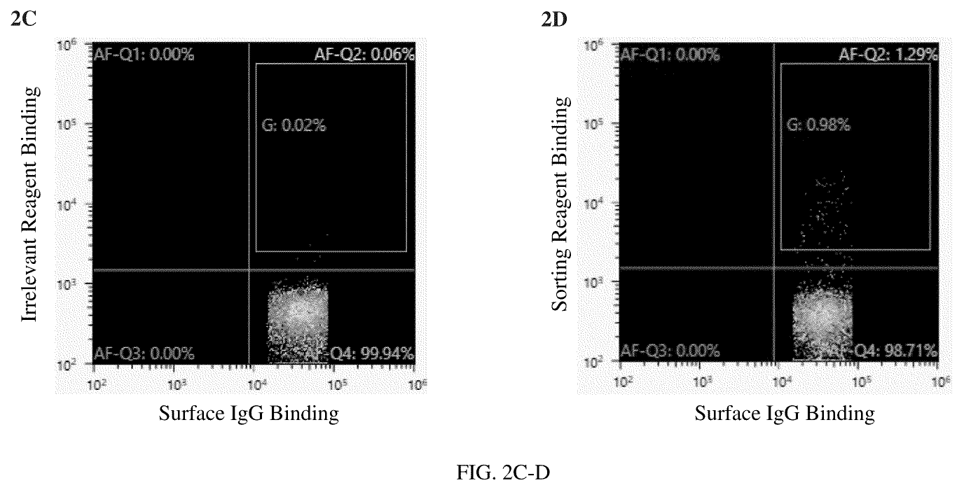

In applying the direct antigen sort method depicted in FIG. 1A, FACS was used to identify and isolate antibody-producing cells that bound to the biotin labeled RBD monomer presented by the biotin/streptavidin-PE tetramer complex by detecting a signal emitted by PE affixed to the complex (FIG. 2A-2B).

In applying the non-compete sort method depicted in FIG. 1B, FACS was used to identify and isolate antibody-producing cells that have bound to the unlabeled RBD monomer which was pre-bound to R001 Fab presented by Alexa fluor 647-labeled R002 by detecting a signal emitted by Alexa 647 affixed to the complex (FIG. 2C-2D).

B cells that were identified through either the direct antigen sort method (FIG. 2B) or the non-compete sort method (FIG. 2D) were individually sorted into 384-well plates, with one cell per well. The individual B cells were subsequently cloned into expression vectors containing human IgG constant regions. Recombinant antibodies were secreted from CHO cells after transient transfection and the antibody-containing conditioned media were collected and ready for further screening assays such as Luminex binding measurement and Octet cross competition assay.

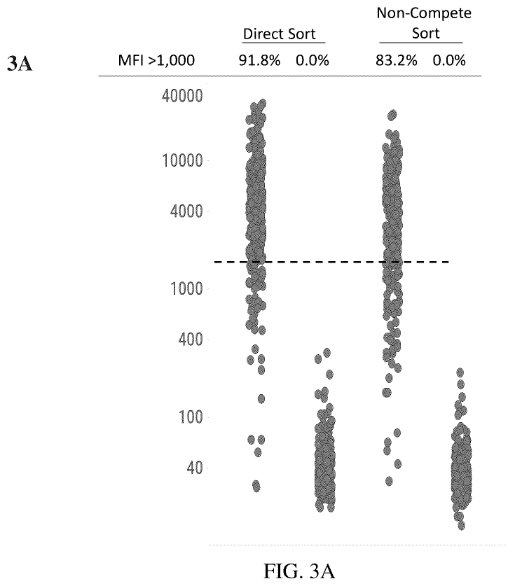

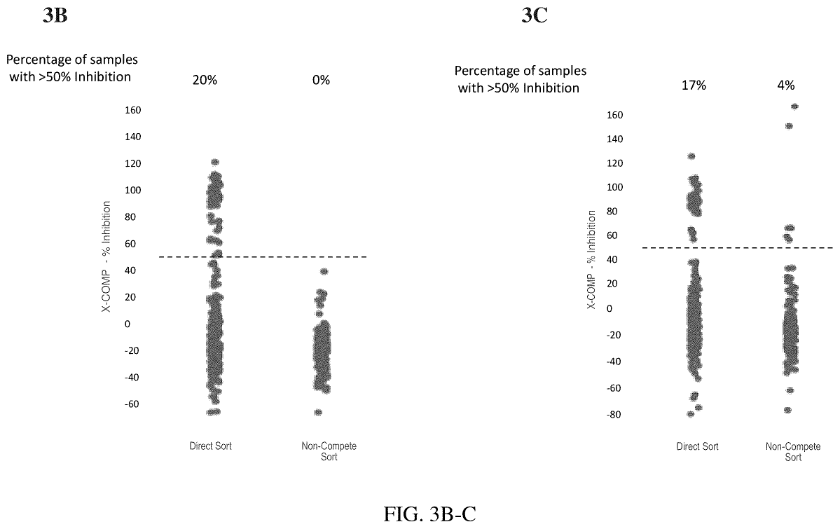

FIGS. 3A-3C present the results from the two sorting methods tested in the following two assays: Luminex antigen binding assay (FIG. 3A) and Octet cross competition assay (FIGS. 3B and 3C). Luminex assay showed that antibody clones from the two sort methods have comparable antigen binding percentages, and Octet cross competition assay demonstrated differential distributions of REGN10933 and REGN10987 non-competing antibodies between the two sort methods.

Luminex assay is a bead-based immunoassay that provides multiplex detection of multiple analytes simultaneously. Recombinant proteins are covalently coupled to paramagnetic Luminex beads (MagPlex beads, Luminex Corp.) for the assay. Biotin labeled proteins or myc-tagged proteins are captured onto Neutravidin (Thermo) or anti-myc goat IgG (Novus) coupled beads respectively for the assay.

For coupling proteins to the beads: beads are first resuspended in 0.1M NaPO4, pH 6.2 (activation buffer), followed by magnetic separation from the buffer. The beads are sequentially added 50 mg/mL N-hydroxysuccinimide (NHS, Thermo Scientific) and 50 mg/mL of 1-ethyl-3-[3-dimethylaminopropyl]carbodiimide (EDC, ThermoScientific) at 1:1 (V:V) at RT. After 10 minutes incubation in dark, the pH of the reaction is reduced to 5.0 with the addition of 50 mM MES, pH 5 (coupling buffer). The beads are vortexed and magnetically separated to remove supernatant and immediately mixed with 20 μg/mL of each protein or 10 μg/ml of Streptavidin or 10 μg/ml anti-myc antibody in coupling buffer and incubated for two hours at RT. For capturing biotin labeled protein or myc-tagged protein onto beads, Neutravidin or anti-myc antibody coupled beads are incubated with biotin labeled or myc-tagged proteins. Beads are then washed with PBS; Neutravidin beads are further blocked with excessive biotin followed by washes with PBS. Biotin labeled protein or myc-tagged protein captured beads are resuspended in PBS 2% BSA 0.05% NaN₃ and ready for assay.

For the immunoassays, beads and diluted Ab samples (like CHO supernatants containing secreted antibodies) are added into each well, and the plates are incubated for two hours at 25° C. and then washed twice with PBS with 0.05% Tween 20. To detect bound antibody levels to individual beads, PE labeled AffiniPure F(ab′)2 Fragment Goat Anti-human IgG, F(ab′)2 Fragment Specific (Jackson Immunoresearch) are added and incubated for thirty minutes at 25° C., followed by two washes. The plates are read in a Luminex FM3D analyzer with xPONENT 4.2 software.

FIG. 3A demonstrates Luminex bindings results from the B cells collected from three SARS-CoV-2 RBD immunized mice using the direct antigen sort method and the non-competing sort method. For the clones from the direct antigen sort method, three hundred and five clones were tested with 2019-n-CoV-2 RBD.mmh protein (“mmh” representing a myc-myc-histidinepeptide; in this case, the myc-myc-histodine is tagged to the C term of the protein), and with irrelevant FelD1.mmh protein. For the clones from the non-competing sort method, two hundred and sixty-three clones were tested with the same two proteins. Here, both methods showed comparable antigen-specific binding percentages to 2019-n-CoV-2 RBD.mmh: 91.8% of the tested clones from the direct antigen sort method had binding mean fluorescence intensity (MFI) greater than 1,000 and were deemed as antigen-specific; 83.2% of the tested clones from the non-competing sort method had binding MFI greater than 1,000. None of the clones from both methods showed specific binding to the irrelevant protein (>MFI 1,000).

Cross Binding Competition was done between the tested anti-SARS-COV-2 monoclonal antibodies and the two antibodies used in the non-compete sorting (antibody R001 and R002) using a real-time, label-free bio-layer interferometry (BLI) assay on an Octet HTX biosensor. To assess whether two antibodies compete with one another for binding to the respective epitopes on SARS-COV-2 RBD, spike protein RBD with his-tag was first captured onto anti-his antibody coated Octet biosensors by submerging the biosensors into solutions containing excessive SARS-CoV-2 RBD.mmh. The antigen-captured biosensors were then saturated with the first anti-SARS-COV-2 monoclonal antibody (subsequently referred to as mAb-1) by immersion into wells containing mAb-1 (R001, R002, or an isotype control R009), followed by dipping the biosensor tips into CHO transient transfected conditioned media containing the test anti-SARS-CoV-2 monoclonal antibodies (mAb-2). The binding responses were monitored and recorded. The responses of mAb-2 bindings to SARS-CoV-2 RBD monomer pre-complexed with R001, R002, or the isotype control were compared. The percentage inhibition by the four known antibodies was calculated using the formula below:

$$Percent {inhibition}^{*} = 100 * ( 1 - ratio of test mAb on mAb - 1 pre - bound sensor to isotype control pre - bound sensor )$$

The calculated percent inhibition of prebound mAb-1 can be used to determine whether the test mAbs (mAb-2) share the same binding epitopes on RBD protein (i.e., compete with each other).

All of the monoclonal antibodies tested in FIGS. 3B and 3C have confirmed anti-SARS-CoV-2 RBD binding (>1,000 MFI) from Luminex measurement shown in FIG. 3A.

FIG. 3B displays the calculated percentage inhibition of R001 on the individual tested anti-SARS-CoV-2 RBD monoclonal antibodies isolated using either the direct antigen sort method or the non-compete sort method. Out of the two hundred and fifty-five antibodies isolated using the direct antigen sort method, fifty-two clones or 20% showed greater than 50% antigen binding inhibition when RBD is pre-bound with R001; while out of the one hundred and seventy-three antibodies isolated using non-competing sort method, none (0%) of them showed greater than 50% inhibition in binding to SARS-CoV-2 RBD that prebound with R001. FIG. 3C demonstrated a similar pattern of the calculated percentage inhibition when RBD is pre-bound with R002. 17% of the antibodies (43 out of 255 clones) isolated using the direct antigen sort method showed greater than 50% inhibition in binding to R002-prebound SARS-CoV-2 RBD; while only as low as 4% of the tested anti-SARS-CoV-2 RBD monoclonal antibodies (7 out of 173 clones) isolated through the non-compete sort method showed greater than 50% inhibition in binding to R002-prebound SARS-CoV-2 RBD.

To conclude, the non-compete sort method significantly reduces SARS-CoV-2 RBD antibodies that compete with R001 and with R002 when compared to the antibodies isolated using direct antigen sort method. The non-compete sort method enriches the isolation of antibodies that are non-competing to the specific targeted antibodies.

Detailed results as mentioned are summarized in TABLE 1.

TABLE 1

| | Count of | # of Abs | # of Abs |
|---|---|---|---|
| | Total Clone | with >50% R001 | with >50% R002 |
| Sort Strategy | Abs | inhibition | inhibition |
| Direct Antigen Sort | 255 | 52 (20.4%) | 43 (16.9%) |
| Non-Compete Sort | 173 | 0 (0.0%) | 7 (4.0%) |

### Example 2: Detecting and Separating Non-Competing Antibodies from Competing Antibodies with Multimeric Antigen

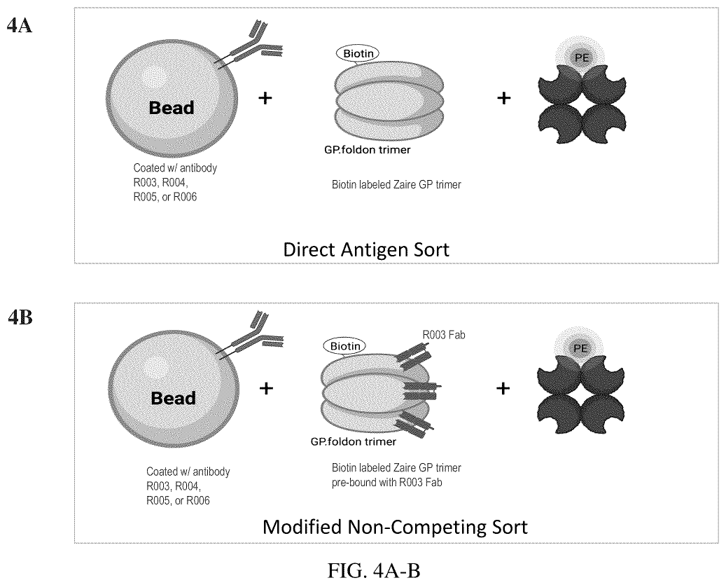

FIG. 4A and FIG. 4B depict the strategies designed for proof-of-concept (POC) experiments for obtaining cells expressing antibodies specific to a multimeric antigen such as the Ebola Zaire glycoprotein (GP) using the direct sort method (FIG. 4A) and the non-compete sort method (FIG. 4B). The Ebola glycoprotein (GP) exists as a homotrimer, and the trimeric structure allows for multiple identical binding sites presented on the surface of the homotrimer.

In both FIG. 4A and FIG. 4B, beads are separately coated with four Ebola Zaire GP antibodies with various levels of competition to a known anti-Ebola GP antibody, R003, as shown in TABLE 1. Four Ebola Zaire GP antibodies were used in these POC experiments:R003, R004, R005 and R006. Table 1 presents the results from Octet cross binding competition assay and illustrates the levels of competition of these four antibodies with R003. As shown in the first row in TABLE 1, R003 fully competes with itself (i.e., identical epitopes on the GP protein) and showed minimal binding at 0.06 nm. R004 partially competes with R003 with low binding at 0.17 nm when GP protein is prebound to R003. Meanwhile, both R005 and R006 showed good binding to R003 prebound GP protein (at 0.68 nm and 0.59 nm, respectively) and exhibit minimal to no cross-binding competition with R003 to the GP protein.

TABLE 2

mAb1/(column)

| mAb2 (row) | R003 | R004 | R005 | R006 |
|---|---|---|---|---|
| R003 | 0.06 | 0.17 | 0.68 | 0.59 |
| R004 | 0.00 | 0.02 | 0.72 | 0.65 |
| R005 | 0.36 | 0.48 | 0.11 | 0.57 |
| R006 | 0.40 | 0.54 | 0.49 | 0.15 |

In the direct sort method as shown in FIG. 4A, beads are first separately coated with the four Ebola Zaire GP antibodies. Antibody-coated beads are contacted with biotin labeled Ebola GP trimer protein. After washing off the unbound, beads are further contacted with a fluorophore (e.g., PE) labeled streptavidin tetramer that forms complex with the biotin on the Ebola GP trimer protein bound to the beads. Beads are then scanned and detected by a fluorescence-based method like Flow Cytometry.

In the non-competing sort method shown in FIG. 4B, antibody-coated beads are contacted with biotin labeled Ebola GP trimer protein that is pre-incubated with excessive Fab of R003. After washing off the unbound, beads are further contacted with a fluorophore (e.g., PE) labeled streptavidin tetramer that forms complex with the biotin on the Ebola GP trimer protein in the presence of excessive Fab of R003. Beads are further scanned and detected by a fluorescence-based method like Flow Cytometry. Alternatively, beads can be contacted with a fluorophore conjugated multimeric antigen directly (instead of through biotin-streptavidin pairing).

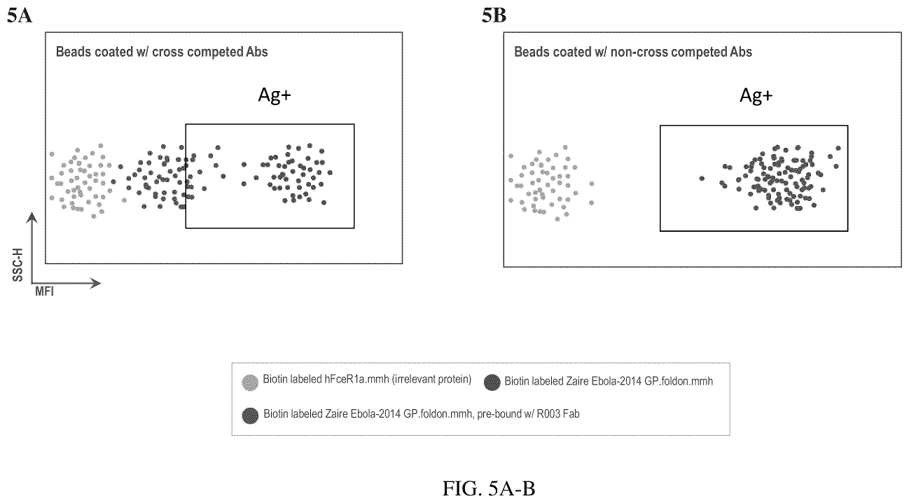

FIGS. 5A and 5B are modeled overlays of PE staining dot plots showing the anticipated results of the above POC experiments.

Both R003 competing antibody-coated beads and R003 non-competing antibody-coated beads are anticipated to exhibit little to no binding after contacting with biotin labeled irrelevant protein and are depicted as the light blue dots outside the “Ag+” box. Both R003 competing and non-competing antibody-coated beads are anticipated to exhibit strong bindings to biotin labeled Ebola GP trimer protein as depicted in the bright blue dots inside the “Ag+” box regardless of which sort methods used. In FIG. 5A, R003-competing antibody coated beads are anticipated to exhibit weakened PE staining level (red dots) and moved outside the “Ag+” box when using the non-competing sort method, due to binding epitopes masked by R003 Fab. In FIG. 5B, R003 non-competing antibody coated beads are anticipated to exhibit unchanged high PE staining level regardless of the sort methods used. This is due to that the antibody coated on beads is not competing with R003 binding epitope on the Ebola GP trimer, and the binding should not be affected by prebinding R003 Fabs onto the Ebola GP trimer (red dots).

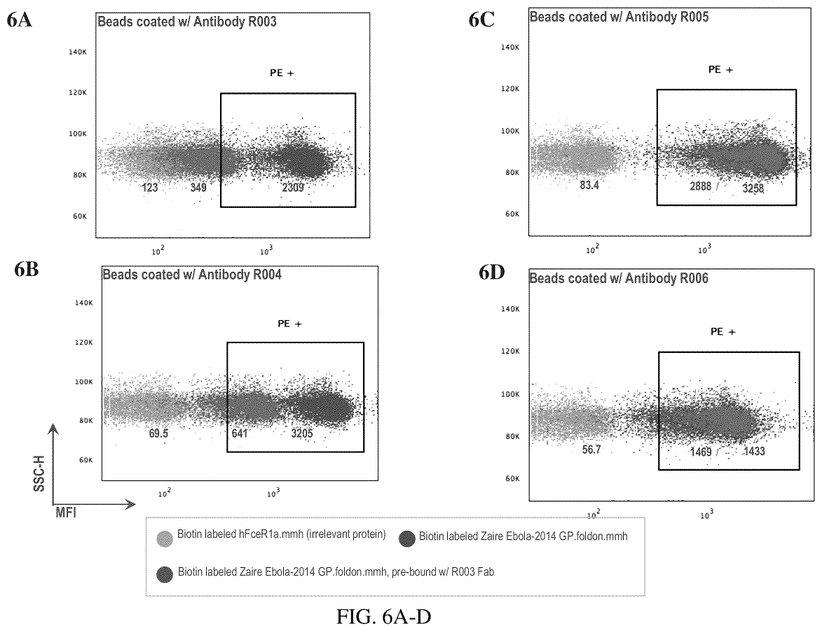

FIG. 6A and FIG. 6B are representative overlays of PE staining dot plots with R003 coated beads and with R004 coated beads detected by flow cytometry, respectively. R003 and R004 are competing antibodies for R003. Three different samples are shown here: light blue dots are beads contacted with a biotin labeled irrelevant protein (control sample) showing PE negative staining (with MFI at 123 and at 69.5 with R003 and R004 coated beads, respectively); dark blue dots are beads contacted with biotin labeled Ebola GP trimer and further stained with PE labeled streptavidin after removing the unbound (Direct Antigen Sort Method), and have exhibited strong PE signals in the positive gates (rectangular boxes) at MFI 2309 and 3205 with R003 and R004 coated beads, respectively; and red dots are beads exposed with R003 Fab-prebound biotin labeled Ebola GP trimer and further stained with PE labeled streptavidin after the removal of the unbound (Non-Competing Sort Method).

R003 and R004 coated beads that underwent the non-competing sort method showed weakened PE signals (red dots) as compared to beads that underwent the direct antigen sort method (dark blue dots). R003 coated beads showed significantly decreased PE signals, and moved down from the positive gate with MFI at 349; R004 coated beads show partially weakened PE signal with MFI at 641. While still largely in the positive gate, R004 coated beads had the PE signal 80% decreased, with MFI from 3205 when the biotin labeled GP trimer was not prebound to 641 when the biotin labeled GP trimer was R003 Fab-prebound. This corresponds to the Octet cross-competition data (TABLE 1) between R004 and R003, in that the GP protein captured biosensor only showed partial R004 loading when first saturated with R003. PE staining from the sample that underwent the non-competing sort method can be clearly separated from the sample that underwent the direct antigen sort method in both cases (i.e. red dots vs. bright blue dots in FIGS. 6A and 6B). These results indicate that with the non-competing sort method, setting a more stringent gate as the positive gate can avoid competing antibodies and enrich for non-competing antibodies.

FIG. 6C and FIG. 6D are representative overlays of PE staining dot plots with R005 coated beads and with R006 coated beads detected by flow cytometry, respectively. R005 and R006 are non-competing antibodies for R003. Three different samples are shown here: light blue dots are beads incubated with a biotin labeled irrelevant protein (control sample) showing PE negative staining (with MFI at 83.4 and at 56.7 with R005 and R006 coated beads, respectively); dark blue dots are beads contacted with biotin labeled Ebola GP trimer and further stained with PE labeled streptavidin after removing the unbound (direct antigen sort method), and have shown strong PE signals in the positive gates (rectangular boxes) at MFI 3258 and 1433 with R005 and R006 coated beads, respectively; and red dots are beads contacted with R003 Fab-prebound biotin labeled Ebola GP trimer and further stained with PE labeled streptavidin after the removal of the unbound (Non-Competing Sort Method).

R005 and R006 coated beads that underwent the non-competing sort method (i.e. red dots) have shown the same level of PE signals as the beads that underwent the direct antigen sort method. R005 coated beads have PE signal at 2888 (MFI) with the non-competing sort method, as comparably strong as it is at 3258 (MFI) with the direct antigen sort method; R006 coated beads show PE signal at MFI 1469 also as comparably strong as it is at MFI 1433 when treated with the direct antigen sort method. Red dots are highly superimposed with the dark blue dots in the PE+ gate in both cases. These results indicate that for non-competing antibody coated beads (either with R005 or R006), signals are not affected by the treatment with R003 Fab-prebound biotin labeled GP trimer and still show strong PE staining in the positive gate.

Taking the observations from FIGS. 6A and 6B (competing antibody coated beads), and from FIGS. 6C and 6D (non-competing antibody coated beads), it is concluded that with the non-competing sort method, competing antibodies give decreased staining signals and non-competing antibodies give comparably unchanged staining signals; and while the extent of decrease may vary, setting a more stringent gate as the positive gate can avoid competing antibodies and enrich for non-competing antibodies.

### Example 3: Detecting Non-Blocking Antibodies Vs Blocking Antibodies with hPCSK9

FIGS. 7A and 7B depict the strategies designed for proof-of-concept experiments for the detection and separation of non-blocking and blocking antibodies to PCSK9 using the non-competing sort method in a dual-color format. Proprotein convertase subtilisin/kexin type 9 (PCSK9) binds to the EGF-like repeat (EGF-A) domain of low-density lipoprotein receptors (LDLR) and prevent LDLR recycling to the cell surface (C G, et al. Acid-dependent ligand dissociation and recycling of LDL receptor mediated by growth factor homology region. Nature. 1987; 326:760-765). Using the non-competing method, one can separate PCSK9 blocking antibodies from non-blocking antibodies and can further enrich for the isolation of either blocking antibodies or non-blocking antibodies.

In this example, human PCSK9 monomer was labeled with a fluorophore, specifically Alexa Fluor 647 (A647). FIG. 7A demonstrates an embodiment of the method wherein antibody-coated beads were contacted with biotin labeled LDLR.mmh and streptavidin-PE sequentially, with washes conducted in between. FIG. 7B demonstrates an embodiment of the method wherein biotin labeled LDLR.mmh was pre-clustered with streptavidin-fluorohpore in 2:1 ratio prior to its addition.

In the experiments depicted in FIG. 7A and FIG. 7B, beads were first coated with PCSK9 blocking antibody R007 or PCSK9 non-blocking antibody R008, and were contacted with A647 labeled hPCSK9 monomer. The antibody coated beads were then further exposed to either biotin labeled LDLR.mmh (FIG. 7A), or biotin labeled LDLR.mmh pre-clustered with streptavidin-PE (in a 2:1 ratio) (FIG. 7B) after the removal of unbound PCSK9. For the method following FIG. 7A, beads were further incubated with streptavidin-PE after removal of the unbound.

Antibody coated beads that underwent both conditions (FIGS. 7A and 7B) can be detected and analyzed through flow cytometry.

FIG. 8A-8C are representative flow staining profiles that demonstrate the detection and separation of the two different anti-PSCK9 antibody coated beads in two different non-competing method conditions as discussed above (FIGS. 8A and 8B). Results from beads coated with R007, a PCSK9 blocking antibody, are shown in FIG. 8A. 99.8% of the population appeared in Q1 quadrant as the A647-positive and PE-negative population, showing that R007 bound to PCSK9 (A647) and prevented PCSK9 from further interacting with LDLR (PE). Results from beads coated with R008, a PCSK9 non-blocking antibody, are shown to have 83.7% in the A647 and PE double positive gate (Q2 quadrant), with a 16.2% tail in the A647 only gate (Q1 quadrant) when biotin labeled LDLR and streptavidin tetramer PE are added sequentially (FIG. 8B), and 99.1% of the total population in the A647 and PE double positive gate (Q2 quadrant), with only a residual tail of 0.85% in the A647 only gate (Q1 quadrant) when biotin labeled LDLR and streptavidin tetramer PE are pre-clustered (FIG. 8C).

The population of blocking antibody (R007 coated beads, FIG. 8A) can be well-separated from the population of non-blocking antibody (R008 coated beads, FIG. 8B and FIG. 8C). Therefore, the non-competing sort method can be applied to enrich for non-blocking population (gating on the double-colored population), as well as for enriching blocking population, i.e. gating on the A647 single positive population.

1. A method for obtaining antibody-producing cells that express antibody molecules to a monomeric target antigen that are non-competitive with a first binder to the target antigen, the method comprising:
(a) contacting a population of antibody-producing cells with:
(1) the target antigen; and
(2) the first binder to the target antigen, wherein the first binder is conjugated with a detectable label;
to permit binding of cells that express antibody molecules that are non-competitive with the first binder to bind to the target antigen; and
(b) collecting cells that are bound to the target antigen which is bound by the first binder conjugated to the detectable label, thereby obtaining a population of antibody-producing cells expressing antibody molecules that are non-competitive with the first binder.

2. The method of claim 1, wherein the antibody producing cells are primary antibody producing cells; and optionally, wherein the primary antibody-producing cells are obtained from the spleen, lymph node, peripheral blood and/or bone marrow of a mammal.

3. (canceled)

4. The method of claim 1, wherein the antibody producing cells are immortalized mammalian cells; and optionally, the immortalized mammalian cells are Chinese hamster ovary (CHO) cells, hybridoma cells, or Human Embryonic Kidney (HEK) 293 cells.

5.-7. (canceled)

8. The method of claim 1, wherein the antibody producing cells are yeast.

9. The method according to claim 1, wherein in step (a), the target antigen is prebound with the first binder prior to being contacted by the population of antibody-producing cells.

10. The method according to claim 1, wherein in step (a):
i. the target antigen is not prebound with the first binder, but is prebound with at least one other binder that does not compete with the first binder in binding to the target antigen, and the contacting permits cells that express antibody molecules that are non-competitive with the first binder and non-competitive with the at least one other binder to bind to the target antigen; and optionally, wherein the at least one binder comprises two or more binders that do not compete with the first binder: or
ii. the target antigen is not prebound with any binder prior to being contacted by the population of antibody-producing cells.

11.-12. (canceled)

13. The method of claim 10, wherein in step (a) the cells are contacted with the target antigen, and subsequently with the first binder conjugated with the detectable label.

14. The method of claim 13, wherein the method further comprises a wash step between contacting with the target antigen and contacting with the first binder; and optionally, wherein the wash step is repeated at least once.

15. (canceled)

16. The method of claim 1, wherein the method further comprises a wash step after step (a) but before collecting cells in step (b); and optionally, wherein the wash step after step (a) and before the collecting step (b) is repeated at least once.

17. (canceled)

18. The method of claim 1, wherein the first binder is an antibody or antigen binding fragment thereof.

19. The method of claim 1, wherein the first binder is a receptor or ligand binding domain thereof, or is a ligand.

20. The method of claim 10, wherein the at least one other binder is an antibody or antigen binding fragment thereof.

21. The method of claim 1, wherein the detectable label conjugated to the first binder is a fluorescent label.

22. The method of claim 14, wherein the detectable label conjugated to the first binder is biotin.

23. The method of claim 10, wherein the at least one other binder is also conjugated with a detectable label; and optionally, wherein the detectable label conjugated to the at least one other binder is a fluorescent label and/or wherein the detectable label conjugated to the at least one other binder is biotin.

24.-25. (canceled)

26. The method of claim 1, wherein the target antigen is a protein; and optionally, wherein:
the protein is an external glycoprotein of a virus; or
the target antigen is a SARS-COV-2 RBD (receptor binding domain) Protein, an Ebola glycoprotein subunit, MERS-COV RBD monomer protein, Malaria sporozoite surface protein, or Proprotein convertase subtilisin/kexin type 9 (PCSK9).

27.-28. (canceled)

29. The method of claim 1, wherein the antibody-producing cells are contacted with the target antigen at a concentration from 0.02 nM to 25 nM.

30. The method of claim 1, wherein the antibody-producing cells are contacted with the first binder conjugated with a detectable label at a concentration from 0.01 nM to 100 nM.

31. A method for obtaining antibody-producing cells that express antibody molecules to a multimeric target antigen that are non-competitive with a first binder to the multimeric target antigen, the method comprising:
(a) contacting a population of antibody-producing cells with the target antigen, wherein the target antigen is conjugated with a detectable label and is prebound by the first binder to permit binding of cells that express antibody molecules that are non-competitive with the first binder to bind to the target antigen; and
(b) collecting cells that are bound to the target antigen that is conjugated with the detectable label.

32. The method of claim 31, wherein the antibody producing cells are primary antibody producing cells.

33.-47. (canceled)

48. The method of claim 31, wherein the protein is an external glycoprotein of a virus; and optionally wherein the protein is an Ebola glycoprotein, an Influenza HA trimer protein, a SARS-COV-1 Spike protein, or a MERS-COV Spike protein.

49. (canceled)

50. A method for obtaining antibody-producing cells that express antibody molecules to a monomeric target antigen that are competitive and/or non-competitive with a first binder to the target antigen, the method comprising:
(a) contacting a population of antibody-producing cells with:
(1) the target antigen conjugated to a first detectable label; and
(2) the first binder to the target antigen, wherein the first binder is conjugated with a second detectable label that is different from the first detectable label;
to permit binding of cells that express antibody molecules that are non-competitive with the first binder to bind to the target antigen; and
(b) collecting cells that are bound to:
(1) the target antigen conjugated to the first detectable label but not the first binder conjugated with the second detectable label, thereby obtaining a population of antibody-producing cells expressing antibody molecules that compete with the first binder in binding to the target antigen; and/or
(2) the target antigen conjugated to the first detectable label and the first binder conjugated to the second detectable label thereby obtaining a population of antibody producing cells expressing antibody molecules that are non-competitive with the first binder in binding to the target antigen.

51. The method of claim 50, wherein the antibody producing cells are primary antibody producing cells.

52.-74. (canceled)
